(19) United States
(12) Patent Application Publication
(10) Pub. No.: US 2011/0256510 A1
(43) Pub. Date: Oct. 20, 2011

(54) Use of Quaternary Ammonium Compounds to Inhibit Endogenous MMPs in Tooth Dentin

(76) Inventors: David Henry Pashley, Augusta, GA (US); Franklin Chin-Meng Tay, Augusta, GA (US); Milena Cadenaro, Trieste (IT)

(21) Appl. No.: 12/741,855

(22) PCT Filed: Jan. 11, 2010
(86) PCT No.: PCT/US2010/020662

§ 371 Date: Apr. 18, 2011

Related U.S. Application Data
(60) Provisional application No. 61/204,669, filed on Jan. 10, 2009.

(51) Int. Cl. A61C 5/04; A61K 31/255; A61K 31/221; A61K 31/14; A61K 6/02; C07C 305/14; C07C 229/30; C07C 233/09; C07C 211/63; A61K 31/38; C07D 335/06

(52) U.S. Cl. 433/228.1; 514/437; 514/517; 514/549; 514/627; 514/642; 549/27; 558/28; 560/222; 564/192; 564/291

(57) ABSTRACT

Disclosed are compositions and methods of using such compositions for inhibiting matrix metalloproteinase activity in dental tissue. The compositions, methods and uses may prevent degradation of the bonding between restorative materials and dental tissues, thereby increasing durability and longevity of the restorative material-dental tissue bonds. For example, the compositions, methods, and uses of the present invention may be used for treating carious dental tissue such as by the creation of dental fillings, crowns, bridges, among other techniques, as well as the creation of esthetic laminate restorations.

### CROSS-REFERENCE TO RELATED APPLICATION

This application claims the benefit of U.S. Provisional Patent Application Ser. No. 61/204,669, filed Jan. 10, 2009, the entirety of which is herein incorporated by reference.

### STATEMENT REGARDING FEDERALLY SPONSORED RESEARCH OR DEVELOPMENT

Some aspects of the invention described in this application were sponsored by DE 015306-06 from the National Institute of Dental and Craniofacial Research. Accordingly, the Federal Government has rights in this application.

### FIELD OF THE INVENTION

The present invention relates to dental compositions for inhibition of matrix metalloproteinases and methods of use. The dental compositions may include quaternary ammonium compounds or biguanide compounds that inhibit matrix metalloproteinases. In some cases, the matrix metalloproteinases inhibited by the compositions and methods of the present invention are dentin collagenases.

### BACKGROUND

Teeth are one of the tissues in the body that undergo biomineralization, the process by which living organisms secrete inorganic minerals in the form of biominerals within body tissue and/or structures. Teeth have four major components: enamel, dentin (or dentine), cementum and pulp. The enamel of a tooth is the intensely hard calcareous (i.e., calcium based) substance that forms a thin layer which caps or partly covers the teeth of most mammals, including humans and other vertebrates. Dentin also comprises calcareous material. It is usually covered by enamel on the crown and cementum on the root and surrounds the entire pulp (i.e., the living tissue of the tooth). Dentin is a living tissue comprised of mineralized matrix p minute tubules which enter into the inner cavity of the tooth where the pulp is housed. The major organic component of dentin is type I collagen. Type I collagen forms a three-dimensional network within which deposition of noncollagenous proteins and the nucleation of hydroxyapatite crystals occur. The cementum is a thin, fairly hard bone tissue covering the root of the tooth.

Although the outer covering of teeth is hard enamel, the largest fraction of teeth is composed of less hard but tougher dentin. Dentin is a biocomposite made up of 50 vol % hydroxyapatite crystallites, 30 vol % collagen and 20 vol % water. The collagen is organized into a collagen fibril meshwork. Each collagen fibril is only 50-100 nanometers in diameter and is separated from its neighbor by a 20 nanometer wide interfibrillar space. When collagen fibrils are secreted developmentally, a number of noncollagenous proteins become bound to the collagen (about 10% of total dentin protein, with collagen being 90% of total dentin protein). These noncollagenous proteins include growth factors (insulin-like growth factors, transforming growth factor-β (TGF-β1, 2, 3), bone morphogenetic proteins (BMPs), acidic and basic fibroblast growth factors, vascular endothelial growth factor (VEGF), a number of regulatory proteins (like dentin phosphoproteins or phosphosphoryns and dentin sialoprotein) and a number of matrix metalloproteinases (MMPs-2, 8, 9, 13, 20).

Endogenous collagen degrading enzymes (collagenases) in dentin are matrix metalloproteinases (MMPs), such as MMP-2, -3, -8, -9 and -20 (Pashley, D. H. et al., J. Dent. Res., 2004, 83:216-221; Tjäderhane, L., et al., J. Dent. Res., 1998, 77: 1622-1629; Sulkala, M., et al., J. Dent. Res., 2002, 81: 603-607; Bourd-Boittin, K., et al., J. Histochem. Cytochem., 2004, 52: 437-45; Mazzoni, A., et al., J. Dent. Res., 2007, 86: 436-440; Mazzoni, A., et al., J. Biomed. Mater. Res. A, 2009, 88: 697-703; Sulkala, M., et al., Arch. Oral Biol., 2007, 52: 121-127; Boukpessi, T., et al., Biomaterials, 2008, 29: 4367-73). MMPs are hydrolases, enzymes that catalyze the addition of water across specific peptide bonds in collagen and gelatin causing severing of one bond. MMPs are the only known mammalian enzymes capable of degrading these collagens. These proteases play a role developmentally (Heikinheimo and Salo, 1995; Bartlett and Simmer, 1999; Tjäderhane et al., 2002; Bourd-Boittin et al., 2005).

During development, once the tooth structure has formed, the collagenous matrix begins to mineralize, causing collagen fibrils to become very stiff due to the growth of nanometer-sized apatite crystallites within (Kinney et al., 2003) and between collagen fibrils (Tay and Pashley, Biomaterials, 2008, 29: 1127-1137). These apatite crystallites cover the noncollagenous proteins in the dentin, including MMPs, making them biologically unavailable and inactive.

Dental caries, also known as tooth decay or cavity, is a disease caused by bacteria in the oral cavity such as Streptococcus mutans and Lactobacilli. Dental caries is a disease wherein bacterial processes damage hard tooth structure (enamel, dentin and cementum). To date, the treatment of dental caries focuses mainly on a surgical model of removing the carious tooth structure followed by replacement with an inert restorative material. Therapy for cavities formed by decay is typically referred to as a “filling” or resin bonding. Dentin bonding is a unique form of tissue engineering in which a demineralized collagen matrix continuous with the underlying mineralized dentin is created via acid-etching or acidic self-etching adhesives and used as the scaffold for resin infiltration. In accordance with this procedure, a dentist or other authorized practitioner may use a drill or a laser to remove the carious dental tissue and may also form undercuts in the tooth structure such that filling material may be secured by the overhang created. The cut surface of the tooth is acid etched and the dentist then fills the cavity with a restorative material to replace the portion of the tooth lost to decay, the restorative material becoming bonded to the tooth tissue. This filling material is placed downward into the tooth from the upper or crown regions of the tooth. Alternatively, the carious dental tissue itself may be acid etched without invasive removal of carious dental tissue prior to application of the restorative material. Restorative material may be adhered to teeth using dental adhesives. Dental adhesives rely on micromechanical entanglement of resin polymers within partially or completely demineralized collagen matrices for retention of the resin composite fillings (Vaidyanathan and Vaidyanathan, J. Biomed. Mater. Res. B Appl. Biomater., 2009, 82:558-578). Infiltration of resins into the demineralized dentin creates a so-called interdiffusion zone or hybrid layer.

Mineralization of the matrix also greatly reduces the permeability of dentin to adhesive monomers making it difficult to bond adhesive resins to dentin (Pashley et al., 2000). This impermeability can be reversed by etching the surface of dentin with any of a number of acidic compounds, including 32-37 wt % phosphoric acid (pH 0.4) or 10-20 wt % acidic methacrylate monomers (pH 1-2.6) for 15-20 sec. These acids rapidly dissolve the apatite crystallites from the dentin matrix to a depth of 1-5 micrometers, thereby exposing the collagen fibrils and all associated noncollagenous proteins, including MMPs. After acid-etching, application of adhesive comonomer mixtures allows the monomers to infiltrate around the collagen fibrils via the interfibrillar spaces. Ideally, the monomers should infiltrate the matrix to the depth that it was demineralized. Unfortunately, such an ideal result is seldom achieved, allowing naked collagen fibrils to remain surrounded by water in water-rich zones.

Despite significant improvements in contemporary resin composites and their bonding to tooth structures via the use of dentin adhesives, it is estimated that half of all resin composite restorations fail within 10 years. Replacement of failed composite restorations accounts for 50-70% of all restorations and replacing them consumes 60% of the dentist's practice time. Secondary caries at the tooth-restoration margins is a major reason for the replacement of existing restorations. Composite-dentin bonds are continuously challenged by the harsh mechanical and chemical environment of the oral cavity, with the risk of secondary caries being 3.5 times higher in resin composite than in amalgam restorations (Bernardo et al., J. Am. Dent. Assoc., 2007, 138:775-783). To treat secondary caries and replace deteriorated dental fillings, additional tooth structure must be removed and/or damaged, which may possibly lead to loss of the diseased tooth. As replacement dentistry costs about 5 billion dollars annually in the United States alone, there is a compelling need to pursue alternative methods to preserve resin-dentin bond integrity and extend the longevity of resin-based restorations.

Unstable bonding of resin-based fillings to teeth is partly due to the proteinaceous nature of dentin, which can result in incomplete infiltration of the resin into the tooth structure. While hydrophilic resin monomers are conventionally thought to be important for bonding of resins to dentin, their inclusion in restorative materials may cause the resulting resin-dentin bonds to be susceptible to degradation via water sorption, leading to what are thought to be the primary causes of resin-dentin bond destabilization: hydrolysis of resin ester linkages and activation of endogenous collagen degrading enzymes (MMPs) (Breschi, L. et al. Dent. Mater., 2008, 24:90-101; De Munck, J. et al., J. Dent. Res., 2005, 84:118-132; Ito, S. et al., Biomaterials, 2005, 26:6449-6459).

Activation of MMPs in dentin may generally arise from acid-etching, as well as by infiltration of acid-producing bacteria beneath restorative materials applied to dental tissue (creating a low pH environment), which exposes the proteins embedded in the collagen matrix (Mazzoni et al., 2006; Nishitani et al., 2006; Tay et al., 2006). Once activated, the MMPs bound to the collagen matrix begin to slowly attack the collagen fibrils that serve to anchor the resin-bonded restoratives to the underlying mineralized dentin (Hashimoto et al., 2000; Pashley et al., 2004; Carrilho et al., 2007). This results in degradation of resin-dentin bonds, reduction in resin-dentin bond strength (Hashimoto et al., 2000; Carrilho et al., 2007), leakage around restorations, development of caries, etc. There is growing evidence that this degradation is due to the hydrolytic activity of endogenous MMPs. For instance, resin-dentin bond strengths fall steadily in vitro when tooth specimens are incubated in water or aqueous media but not when they are incubated in water-free solutions like oil. (Carrilho, M. R. O. et al, Dent. Mater., 2005, 21: 232-241). In addition, studies with MMP-inhibitors suggest that they may prove efficacious in dental restoration preparations.

The current approach to prevent progressive deterioration of dental restorations focuses on the use of antimicrobial agents incorporated into restorative materials. Resin-based restorative materials exhibiting antimicrobial activity have been developed for various compounds using polymerizable cationic monomers that are covalently bound within the polymer matrix (Imazato, S., et al., J. Dent., 1998, 26: 267-271; Imazato, S., et al., J. Biomed. Mater. Res., 1998, 39: 511-515). The antimicrobial activity of quaternary ammonium compounds and biguanides generally requires that such compounds also contain a long, flexible, hydrophobic alkyl chain (C₁₂-C₂₂) that can function to penetrate bacterial cell walls (Domagk, G., Dtsch Med. Wochenschr., 1935, 61: 829-832; Tomlinson et al., J. Med. Chem., 1977, 20: 1277-1282; Pernak et al., Eur. J. Med. Chem., 2001, 36: 899-907). A similar approach for compounds with anti-MMP activity may be useful (Ikeda et al., 1994; Luthra and Sandhu, 2005).

Thus, development of compositions and methods of stabilizing resin-dentin bonds and preventing the endogenous proteolytic degradation of dentin based on the use of MMP-inhibitors in therapeutic restorative materials or therapeutic dental primers is highly desirable.

### BRIEF SUMMARY

Embodiments of the present invention relate to compositions and methods of using compositions suitable for dental use that inhibit matrix metalloproteinase activity in dental hard tissue thereby increasing the durability and longevity of dental composition-dental bonds. The present invention may be embodied in a variety of ways. In some embodiments, the present invention comprises a composition comprising polymerizable quaternary ammonium monomers capable of inhibiting matrix metalloproteinase activity in dental tissue. In certain embodiments, the compound comprises a compound of Formula I as disclosed herein.

In certain embodiments, the present invention comprises a method of inhibiting matrix metalloproteinase activity in dental tissue in a subject comprising administering to the subject a dental composition comprising polymerizable quaternary ammonium monomers of Formula I as disclosed herein.

In other embodiments, the present invention comprises a method of repairing dental caries suing a compound of Formula I as disclosed herein.

In yet other embodiments, the present invention comprises a use of a compound of Formula I as disclosed herein for preparation of a medicament for the treatment of various purposes such as the treatment of tooth decay.

In another embodiment, the present invention comprises a use of a compound of Formula I as disclosed herein for treatment of various diseases such as tooth decay.

In certain embodiments, the compositions and methods may be used to inhibit endogenous MMPs in normal or carious dentin that are activated by the therapeutic acid-etching step in the resin-bonding process by infiltration of the dentin tissue with an anti-MMP monomer that copolymerizes with typical dental adhesive monomers, thereby preventing weakening of underlying sound dentin and increasing durability of dental composition-dental bonds.

The invention has several advantages over the current dental restoration compositions and methods. The compositions and methods of the present invention result in extended durability of resin-dentin bonds by preventing the slow, steady fall in resin-dentin bond strength over time that is caused by endogenous matrix metalloproteinases degradation of the collagen matrix. One advantage is that the application of the dental compositions of the present invention at the time of acid-etching or immediately thereafter in activates MMPs in the dentin collagen matrix that are exposed and activated by the acid-etching process. This prevents these MMPs from hydrolytically cleaving the type I collagen found in the dentin collagen matrix over time, which would compromise the bonding of the dental composition to the dentin.

An additional advantage of the present invention is that the compositions and methods may also prevent degradation of the resin-dentin bond by inactivating MMPs exposed and activated through the action of bacteria present under the dental restoration. In the repair of a tooth containing a dental carie, even after removal of most of the soft, decayed dentin, there is a high probability that the cavity remains contaminated with residual bacteria that may continue to invade sound dentin beneath a restoration. Acid produced by these bacteria causes demineralization of mineralized dentin, thereby softening it and weakening it. This acidic demineralization also exposes and activates MMPs within the dentin collagen matrix, which leads to further degradation of the softened collagen matrix. The quaternary ammonium-containing compositions and methods of the present invention prevent additional collagen matrix degradation due to bacteria-induced exposure of MMPs beneath dental restorations, thereby increasing the durability and longevity of the restoration.

Another advantage of the present invention is that, due to the use of polymerizable agents having anti-MMP activity that can copolymerize with typical dental adhesives in the compositions and methods, the compositions and methods of the present invention will act to inhibit MMP degradation of the resin-dentin bond and increase durability over the life of the restoration. This is in contrast to some current dental compositions in which active agents (e.g., antimicrobial) are not polymerizable and may leach out of the restoration over time.

The advantages of the invention also extends to healthy dentin exposed by dentists during creation of a variety of dental restorations (e.g., esthetic laminate restorations, crowns or bridges, etc.). Apart from restoring caries, dentists may place the invention on healthy dentin after etching of the latter as a means to reduce dentin hypersensitivity, or as a means of coupling an esthetic restoration such as an esthetic resin composite or ceramic veneer to the exposed dentin.

### BRIEF DESCRIPTION OF THE DRAWINGS

FIG. 1 shows, in accordance with alternate embodiments of the present invention, electrophoretic analysis of Clostridium histolyticum collagenase activity in the presence of various known antibacterial compounds to identify compounds with anticollagenolytic activity.

FIG. 2 shows, in accordance with alternate embodiments of the present invention, exemplary electrophoretic analysis of Clostridium histolyticum collagenase activity in the presence of chlorhexidine diacetate (CHX) (Panel A) and [2-(methacryloyloxy)ethyl] trimethyl ammonium chloride (METMAC) (Panel B) for quantification of anticollagenolytic activity.

FIG. 3 shows, in accordance with alternate embodiments of the present invention, a graph illustrating percent loss of dry weight mass (collagen degradation) of demineralized dentin tissue incubated in the presence of various anti-MMP quaternary ammonium compounds for 12 hrs at 25° C. in an artificial saliva buffer.

FIG. 4 shows, in accordance with alternate embodiments of the present invention, a graph showing quantification of solubilized collagen (hydroxyproline) released from demineralized dentin tissue incubated in the presence of various anti-MMP quaternary ammonium compounds for 12 hrs at 25° C. in artificial saliva buffer.

FIG. 5 shows, in accordance with alternate embodiments of the present invention, a schematic of an apparatus that can be used to measure degree of conversion of quaternary ammonium methacrylate resin composition, showing a Mylar film overlaid above an experimental resin film which in turn is overlaid on top of a diamond-attenuated total reflectance (ATR) element mounted horizontally in an FTIR spectrometer.

FIG. 6 shows, in accordance with alternate embodiments of the present invention, an exemplary absorption spectra of an FTIR spectral analysis method depicted in FIG. 5.

### DETAILED DESCRIPTION

Embodiments of the present invention provide compositions and methods of using such compositions to inhibit matrix metalloproteinases in dental hard tissue. Numerous modifications and adaptations are apparent to those skilled in the art without departing from the scope of this disclosure. As set forth above, there is a need for the development and use of dental compositions that may be used in dental restoration materials that can remain bonded to dental tissue for longer periods of time.

Unless indicated to the contrary, the numerical parameters set forth in the following specification are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

It is further noted that, as used in this specification, the singular forms “a,” “an,” and “the” include plural referents unless expressly stated and unequivocally limited to one referent. The term “or” is used interchangeably with the term “and/or” unless the context clearly indicates otherwise.

Also, where ranges are provided, it is understood that other embodiments within the specified ranges are to be included, unless specifically indicated otherwise. If a particular term used is not specifically defined, such term should not be considered indefinite. Rather, terms that are used should be given their ordinary meanings unless it is apparent from the context that another meaning(s) may be intended.

As used herein, a “subject” or an “individual” may be an animal. For example, the subject or individual may be a mammal. Also, the subject or individual may be a human. The subject or individual may be either a male or a female.

As used herein, a “patient” is a human who is under dental or medical care and/or actively seeking dental or medical care for a disorder or disease.

As used herein, “administer” or “administering” refers to the use of a restorative material and/or primer in the preparation of dental tissue in an individual's tooth or teeth for the treatment of dental caries or correction of dental tissue damage.

As used herein, “dental tissue” is tissue that is derived from, or part of, a tooth, such as, for example, enamel, dentin or cementum, that is mineralized or is demineralized due to acid-etching or dental caries.

As used herein, “dentin” is a hard dental tissue comprised of water, organic and inorganic matter, with the inorganic matter comprising primarily hydroxyapatite. The organic matter of dentin is comprised of type I collagen (about 90 wt % of dentin extracellular matrix) and non-collagenous proteins, which provide flexibility and tensile strength. The inorganic material in dentin provides compressive strength and rigidity.

The “type I collagen” refers to a long, fibrous structural protein that is present in several different tissues including, but not limited to, dentin, tendon, bone, lung, skin, heart valves, fascia, scar tissue, cornea, and liver.

As used herein, the “collagen fibers” refers to bundles of collagen fibrils.

As used herein, the “collagen fibrils” refers to individual collagen peptides that self-assemble into triple helical collagen molecules that, in turn, aggregate to form collagen fibrils. The molecules may aggregate with a ¼ overlap to create distinct gaps, sometimes called hole zones.

As used herein, the “tropocollagen” or “collagen molecule” refers to a subunit of larger collagen aggregates such as collagen fibrils.

As used herein, the terms “type I collagen matrix” or “collagen matrix” refers to native or reconstituted aggregations of type I collagen molecules forming a fibrillar scaffold. In dentin, hydroxyapatite (having a modulus of elasticity of about 100,000 MPa) generally forms within the interfibrillar and intrafibrillar spaces of collagen fibrils, which mechanically stiffens the collagen matrix (Wagner and Weiner, J. Biomech., 1992, 25: 1311-1320).

As used herein, “noncollagenous proteins” are proteins bound within the collagen matrix that are not type I collagen. For example, noncollagenous proteins may include highly phosphorylated anionic proteins such as phosphosphoryn and dentin matrix protein 1 (DMP1) that bind to collagen close to the gap zones (Beniash et al., J. Struct. Biol., 2000, 132:212-225; Gajjeraman et al., J. Biol. Chem., 2007, 282:1193-1204; George and Veis, Chem. Rev., 2008, 108:4670-4693). Noncollagenous proteins also include dentin matrix metalloproteinases such as, for example, MMP-2, -3, -8, -9 and -20.

As used herein, “matrix metalloproteinases” are metal-dependent endopeptidases (such as zinc containing endopeptidases) capable of degrading extracellular matrix proteins.

As used herein, “collagenases” are MMPs capable of degrading triple-helical fibrillar collagens (e.g., Type I collagen) into distinctive ¾ and ¼ fragments.

As used herein, “restorative material” is a material that is adhered to dental tissue to create dental fillings and/or other restorative dental work. Restorative material may be, for example, a resin or a cement.

As used herein, “acid-etch” refers to methods and/or compositions related to removing a surface of dental tissue using an acid.

As used herein, “demineralized dental tissue” is dental tissue that has a diminished amount of hydroxyapatite within the tissue compared to normal dental tissue. For example, demineralized dentin has diminished amounts of hydroxyapatite within the type I collagen matrix. Demineralized dental tissue may be carious dental tissue. Demineralized dental tissue may also be acid-etched dental tissue that has been treated with acid in preparation of creating a filling to treat dental caries.

As used herein, “hybrid layer” is an area of a dental tissue directly adjacent to an applied restorative material where the restorative material has bonded to dental tissue components (e.g., dentin extracellular matrix).

As used herein, “adhesive layer” is a layer (2-50 μm thick) of excess bonding resin adjacent to the hybrid layer but not infiltrated into the acid-etched dentin collagen matrix, which may act to couple the overlying resin composite with the underlying hybrid layer.

As used herein, “water-rich zones” or “water-filled voids” or “water-filled channels” are spaces in the hybrid layer where a restorative material and the underlying dental tissue are not bonded to each other that are susceptible to water infiltration.

As used herein, “cement mixture” or “cement” is a mixture of inorganic and/or organic components that, when mixed with a liquid such as, for example, water, forms a paste that subsequently sets or hardens into a solid cement structure.

As used herein, a “primer” is a neutral blend of hydrophilic and/or hydrophobic resin commoners that are dissolved in a solvent to reduce viscosity. It is applied to acid-etched dentin to improve the wettability of the dentin, creating a chemical and micromechanical bond to dentin. Primers may copolymerize with restorative materials such as, for example, resins.

As used herein, a “self-etching primer adhesive” is a solvated resin comonomer blend that contains a sufficiently high concentration of acidic resin monomers to enable the primer to etch through smear layers into the underlying intact dentin or enamel without the use of an acidic etchant. After the application of the acidic primer, a solvent-free adhesive is applied to the primed tooth substrate for coupling the primer to a resin-based restorative material.

As used herein, an “all-in-one self etching adhesive” is a solvated adhesive that combines the self-etching primer and the adhesive into a single component for the sake of increasing the user friendliness and ease of application of the adhesive.

As used herein, “methacrylates” are esters of methacrylic acid. Methacrylates contain methyl-vinyl groups, that is, two carbon atoms double bonded to each other, directly attached to a carbonyl carbon, and wherein the vinyl group is substituted with a non-terminal methyl group. Methacrylates are monomers that may form polymers because the double bonds are very reactive. They have the general chemical formula

CH₂═CMeCOOR

As used herein “acrylates” are the salts and esters of acrylic acid. Acrylates contain vinyl groups, that is, two carbon atoms double bonded to each other, directly attached to a carbonyl carbon. Acrylates are monomers that easily may form polymers because the double bonds are very reactive. They have the general chemical formula

CH₂═CHCOOR

As used herein, “degree of conversion” or “degree of cure” or “conversion degree” or “percent conversion” or “DC” or “PC” describes the percentage of double bonds present in a solution that react when unsaturated monomers are polymerized into saturated polymers.

As used herein, “therapeutic monomers” or “anti-collagenolytic monomers (AC)” are polymerizable monomers capable of forming a polymer that also have a biologic activity, such as, for example, antibacterial, MMP inhibition, or growth stimulation activity.

As used herein, “anti-matrix metalloproteinase activity (anti-MMP)” or “anti-collagenolytic activity” is the ability of a compound to reduce the proteolytic activity of one or more matrix metalloproteinases, including collagenases. For example, such anti-MMP activity may be referred to in terms of percent reduction of proteolytic activity.

As used herein, “acid etching” refers to a process wherein a tooth is prepared for a further dental procedure such as dental fillings and repair of damaged dental tissue that may improve adherence of restorative materials to the tooth. The process involves the demineralization of the dental tissue. The process includes using an acidic solution on a tooth such as a decayed tooth prior to dental restorations that may produce a 5-8 μm thick layer of mineral-free collagen matrix on the surface of the mineralized dentin base. Acid etching may be performed using an acidic etching solution for approximately 15-60 seconds, after which the etching solution may be rinsed thoroughly from the dental tissue with water, and the surface of the tooth then dried for further dental procedures.

As used herein, the “acidic etching solution” refers to an acidic solution that is used in the acid etching process. As an example, acid etching solutions may contain phosphoric acid at a concentration of approximately 37% to 50% (w/v) in a solution or in a gel compound, or may contain a phosphoric acid concentration of approximately 0.51% to approximately 5.40%.

For example, a dental cement may be dispensed as a solid and a liquid that is mixed where the powder may be polymethyl methacrylate, a filler, plasticizer, and polymerization initiator, and the liquid monomer may be methyl methacrylate with an inhibitor and/or an activator to control polymerization. Or, other polymers may be used. Polymerization may occur by chemical initiation or light-activation (for example, blue light activation). The resin-based cement or adhesive may have a cement mixture incorporated into it. For example, the cement mixture may be a calcium silicate and/or calcium phosphate-containing resin cement such that when the cement hardens it releases calcium hydroxide (Ca(OH)₂) (e.g., Portland cement). The cement mixture may be incorporated into a resin or filler composition to form a resin cement.

The various embodiments of the invention will be generally described now with reference to the various figures and examples. A more detailed description appears below. The figures and/or examples are in no way meant to limit the present invention but are to illustrate various exemplary embodiments of the present invention.

The methods of the present invention are described herein with primary reference to the dental industry.

It has been discovered that certain quaternary ammonium compounds (and biguanide compounds) inhibit dentin matrix metalloproteinases (MMPs) as described below, and thereby stabilize the adhesive interface of dental composites over time. For example, the addition of polymerizable quaternary ammonium compounds to various adhesive comonomer mixtures can inhibit the endogenous MMPs in dentin during bonding of the restorative material to the dental tissue and may continue to inhibit those MMPs in the long term. Such stabilized resin-dentin bonds may be more durable and need less replacement of restorations, saving money and discomfort. Similar to CHX (chlorhexidine), quaternary ammonium salts are positively charged at physiological pHs and have effective antibacterial activities (Dizman, B., et al., J. Appl. Polym. Sci., 2004, 94: 635-642; Dizman, B. et al., J. Appl. Polym. Sci. Part A Polym. Chem., 2006, 44: 5965-5973; Ayfer, B., et al., Des. Monomers Polym., 2005, 8: 427-451; Xiao, Y-H, et al., J. Oral Sci., 2008, 50: 323-327; Gomes, B P F A, et al., J. Dent., 2009, 37: 76-81; Namba, N., et al., Dent. Mater., 2009, 25: 424-430; Li., F. et al. 2009). The present invention is based at least in part on the discovery that certain of these quaternary ammonium salts are also good inhibitors of MMPs.

Thus, embodiments of the present invention comprise compositions, methods and uses for restorative dental approaches using anti-matrix metalloproteinase (MMP) quaternary ammonium compounds (QACs) to prevent degradation of restorative resin-dentin bonds that bond resin composites, crowns, bridges, etc to tooth substrates. Endogenous MMPs in dentin, such as, for example, MMP-2, -3, -8, -9 and -20, are present as noncollagenous proteins in the type I collagen matrix. These proteins function as collagenases and, when activated, for example, by acid-etching, they hydrolytically degrade type I collagen.

Various embodiments of the present invention are disclosed herein. It will be understood that the various embodiments as described and claimed herein generally each apply to the compounds, compositions, methods and uses of the present invention.

In certain embodiments, the present invention comprises compositions comprising polymerizable quaternary ammonium monomers capable of inhibiting matrix metalloproteinase activity in dental tissue. The composition may, in certain embodiments, be a dental composition.

Also in various embodiments, the dental compositions of the present invention may include cements, resins, primers, adhesives and mixtures thereof.

In an embodiment, the composition comprises a polymerizable quaternary ammonium monomers that are compounds of Formula I

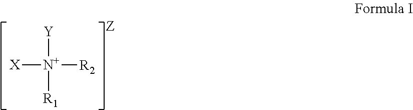

wherein X is selected from the group consisting of —R₄—O—C(O)C(R₅)═CH₂, —CH₂—CH═CH₂, and

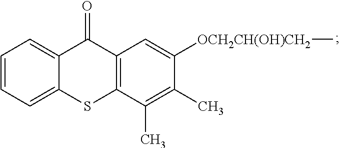

wherein Y is selected from the group consisting of hydrogen, methyl, and —CH₂—CH═CH₂;
or X and Y together with the nitrogen atom to which they are attached form a five membered ring with X and Y having the structure —CH₂—C(R₆)H—C(R₇)H—CH₂—;
wherein Z is a counterion sufficient to balance the charge of the monomer;
wherein R₁ is selected from the group consisting of hydrogen and C₁₋₂alkyl;
wherein R₂ is selected from the group consisting of hydrogen and C₁₋₂alkyl;
wherein R₄ is C₂₋₃alkylene;
wherein R₅ is selected from the group consisting of hydrogen and methyl;
wherein R₆ is an ethylene, acrylate or methacrylate group; and
wherein R₇ is an ethylene, acrylate or methacrylate group.

In alternate embodiments,
Y is selected from the group consisting of methyl, and —CH₂—CH═CH₂;
Z is a counterion selected from the group consisting of a halo group, a methyl sulfate group, or an acetate group;
R₁ is methyl;
R₂ is methyl;
R₄ is ethylene; and
R₅ is selected from the group consisting of hydrogen and methyl.

In another embodiment, the polymerizable quaternary ammonium monomers are compounds of Formula Ia

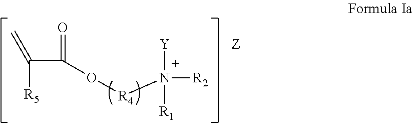

wherein R₄ is ethylene and wherein R₅ is a methyl group.

In some embodiments, the composition may contain compounds that are selected from the group consisting of [2-(methyl-acryloyloxy)ethyl-N-trimethyl ammonium chloride (METMAC), 2-(methacryloyloxy)ethyltrimethylammonium methyl sulfate (MCMS), 2-Acryloxyethyltrimethylammonium chloride (ATA), diayllyldimethyl ammonium chloride (DDAC), 3-[3,4-dimethyl-9-oxo-9H-trioxanthen-2-yloxy]-2-hydroxypropyl] trimethyl ammonium chloride (OTX), [3-(methacryloylamino) propyl]trimethylammonium chloride (MERQUAT 106), and N—N-dimethylaminomethacrylate methyl chloride and mixtures thereof.

In yet other embodiments, the composition further comprises chlorhexidine diacetate (CHX). The above-mentioned polymerizable quaternary ammonium monomers may form a copolymer with CHX methacrylate.

In certain embodiments, the concentration of the compound of Formula I may be within a certain range so as to inhibit MMPs. This concentration range may be distinct from the concentration that may be needed for anti-bacterial effects. In an embodiment, the polymerizable quaternary ammonium monomers may be present in an amount from about 0.2 to 40% by weight. In a variation, the polymerizable quaternary ammonium monomers may be present in an amount from 1.0 to 20% by weight. In a further variation, the polymerizable quaternary ammonium monomers may be present in an amount from 3.0 to 20% by weight. In a further variation, the polymerizable quaternary ammonium monomers may be present in an amount from 5.0 to 15% by weight. Or, ranges within these ranges may be used.

In an embodiment, the present invention also relates to methods and uses of using the compositions of the invention.

Accordingly, in one embodiment, the present invention relates to a method of inhibiting matrix metalloproteinase activity in dental tissue in a subject comprising administering to the subject a dental composition comprising polymerizable quaternary ammonium monomers of Formula I

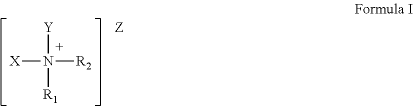

wherein X is selected from the group consisting of —R₄—O—C(O)C(R₅)═CH₂, —CH₂—CH═CH₂, and

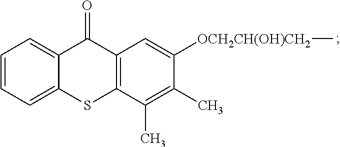

wherein Y is selected from the group consisting of hydrogen, methyl, and —CH₂—CH═CH₂;
or X and Y together with the nitrogen atom to which they are attached form a five membered ring with X and Y having the structure —CH₂—C(R₆)H—C(R₇)H—CH₂—;
wherein Z is a counterion sufficient to balance the charge of the monomer;
wherein R₁ is selected from the group consisting of hydrogen and C₁₋₂alkyl;
wherein R₂ is selected from the group consisting of hydrogen and C₁₋₂alkyl;
wherein R₄ is C₂₋₃alkylene;
wherein R₅ is selected from the group consisting of hydrogen and methyl;
wherein R₆ is an ethylene, acrylate or methacrylate group; and
wherein R₇ is an ethylene, acrylate or methacrylate group.

In a variation of the above embodiment, Y is selected from the group consisting of methyl, and —CH₂—CH═CH₂;
wherein Z is a counterion selected from the group consisting of a halo group, a methyl sulfate group, or an acetate group;
wherein R₁ is methyl;
wherein R₂ is methyl;
wherein R₄ is ethylene; and
wherein R₅ is selected from the group consisting of hydrogen and methyl.

In an embodiment, the method uses the polymerizable quaternary ammonium monomers of Formula Ia:

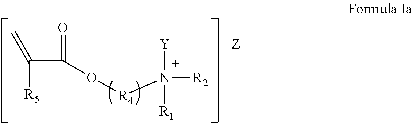

wherein R₄ is ethylene and wherein R₅ is a methyl group.

In an embodiment, the present invention relates to methods of creating better bonding (e.g., longer-lasting, more durable) than currently achievable between a dental cement, primer, adhesive, or other dental composition to a mineralized part of the teeth such as dentin.

In an embodiment, the methods can use any of a variety of dental materials, which may be in a variety of forms including forms comprising acid-etching solutions, primers, adhesives, or cements, or mixtures thereof.

In a variation of the above-embodiment, the dental materials may be any of a plurality of over the counter products such as mouthwashes, toothpastes, or any other oral product, or mixtures thereof.

In an embodiment, the methods use composition amounts that are sufficient to produce the desired result of more durable, long-lasting resin-dentin bonding of restorative materials. In one embodiment, the polymerizable quaternary ammonium monomers are present in the composition in concentrations from about 0.2 to 40% by weight. In a variation, the polymerizable quaternary ammonium monomers may be present in an amount from 1.0 to 20% by weight. In a further variation, the polymerizable quaternary ammonium monomers may be present in an amount from 3.0 to 20% by weight. In a further variation, the polymerizable quaternary ammonium monomers may be present in an amount from 5.0 to 15% by weight. Alternatively, ranges within these ranges may be used.

In an embodiment, the composition of the present invention may be used in methods wherein the composition is an all-encompassing composition that can perform any of a plurality of functions. For example, rather than having a dentist or other practitioner drill a tooth to address a cavity, employ one composition for performing acid etching of the tooth, employ a second composition as a primer, and employ a third composition for filling the tooth, the dentist may use one composition that can perform any two or more of these steps. For example, a dental composition that is used for acid etching a tooth might also contain the appropriate polymeric materials as discussed herein that will form the polymers able to prime and fill teeth, yet have the advantages discussed herein such as being able to inhibit MMPs (matrix metalloproteinases) and consequently, provide longer lasting fillings.

For example, in alternate embodiments, and consistent with the description above, the dental composition may be a part of a self-etching primer, a non-self-etching adhesive, or an all in one self-etching primer adhesive.

In an embodiment, a primer of the instant invention that contains the quaternary ammonium salts of the invention in an embodiment is generally somewhat acidic, such that the primer may be used for acid-etching a tooth. The acidic pH also allows for the quaternary ammonium compounds to remain positively charged. In an embodiment, the pH of the primer may be between about 0.1 to 4.0. Alternatively, the pH of the primer may be between about 0.5 to 4.0. Alternatively, the pH of the primer may be between about 0.5 to 3.8. Alternatively, ranges within these ranges may be used.

Accordingly, in an embodiment, the dental compositions of the present invention may further comprise a photopolymerizable initiator and one or more of the following: (i) a polymerizable monomer containing an acid group, (ii) a polymerizable monomer selected from the group consisting of pyridinium bases and phosphonium bases, (iii) a hydrophilic polymerizable monomer, (iv) a hydrophobic polymerizable monomer, and (v) a polymerizable dimethacrylate monomer.

In certain embodiments, a photopolymerization initiator may be used together with the quaternary ammonium compounds of the present invention in restorative materials to cause polymerization of the quaternary ammonium methacrylates, thereby hardening the restorative material from a liquid or gel mixture into a solid. In certain embodiments, a plurality of photopolymerizable initiators may be used. In one embodiment, the photopolymerizable initiator is TPO (diphenyl (2,4,6-trimethylbenzoyl)-phosphine oxide). In another embodiment, the polymerizable initiator is benzoyl peroxide. In a further embodiment, TPO and benzoyl peroxide may be used together.

In an embodiment, certain inert or ion-releasing fillers may also be used.

In an embodiment, the various compositions may be part of a kit that may aide the user to make the dental compositions of the present invention. The kits may include other oral devices that can be used to generate the dental compositions.

A variety of methods can be used that are known to activate the photopolymerizable initiator(s). For example, in certain embodiments of the present invention, photosensitizers may be used. In a variation, a photopolymerizable initiator may be activated to facilitate polymerization, such as the use of blue light (ca. 450 nm) or UV light (ca. 340 nm). Alternatively, chemical free-radical polymerization, or cationic polymerization may be used. In a variation of these embodiments, more than one photoinitiator may be used. Sometimes the dental adhesive may be placed inside a metallic crown that can not pass blue light (i.e., can not photopolymerized by irradiation) (for example, by visible or UV light). Consequently, in these instances it may be preferable to use a photoinitiator combined with a chemical polymerization initiator.

In an embodiment, the polymerizable initiator may be a chemical initiator (i.e., an initiator that does not require electromagnetic radiation to initiate polymerization).

In an embodiment, the dental composition may comprises a member from each of the following groups: (i) a polymerizable monomer containing an acid group, (ii) a polymerizable monomer selected from the group consisting of pyridinium bases and phosphonium bases, (iii) a hydrophilic polymerizable monomer, and (iv) a polymerizable dimethacrylate monomer.

In an embodiment, the dental composition further comprises a photopolymerization inhibitor. The photopolymerization inhibitor may perform the function of preventing premature, inappropriate polymerization of the blend during storage thereby preventing it from being dispensed from its container. In an embodiment, the photopolymerization inhibitor may be selected from the group consisting of hydroquinone, hydroquinone monomethyl ether and hydroquinone monoethyl ether, or mixtures thereof.

In an embodiment, the dental composition may contain the relevant liquids to perform the desired tasks. For example, as discussed herein, water or various acids may be needed in the dental compositions of the present invention to prepare the tooth and/or provide the appropriate chemical environment for polymerization and/or anti-MMP activity.

In an embodiment, the quaternary ammonium-containing polymers used in the dental compositions of the present invention can be made by following the procedures outlined in Dizman et al., 2004, Dizman et al., 2006; Ayfer et al. 2005; Xiao et al., 2008.

In an embodiment, the present invention includes methods of fixing a dental carie in a tooth comprising the step of administering a dental adhesive wherein the dental adhesive comprises a compound of Formula I

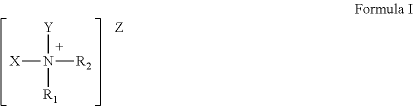

wherein X is selected from the group consisting of —R₄—O—C(O)C(R₅)═CH₂, —CH₂—CH═CH₂, and

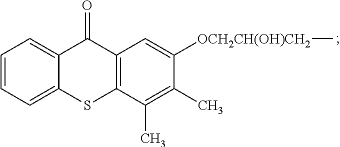

wherein Y is selected from the group consisting of hydrogen, methyl, and —CH₂—CH═CH₂;
or X and Y together with the nitrogen atom to which they are attached form a five membered ring with X and Y having the structure —CH₂—C(R₆)H—C(R₇)H—CH₂—;
wherein Z is a counterion sufficient to balance the charge of the monomer;
wherein R₁ is selected from the group consisting of hydrogen and C₁₋₂alkyl;
wherein R₂ is selected from the group consisting of hydrogen and C₁₋₂alkyl;
wherein R₄ is C₂₋₃alkylene;
wherein R₅ is selected from the group consisting of hydrogen and methyl;
wherein R₆ is an ethylene, acrylate or methacrylate group; and
wherein R₇ is an ethylene, acrylate or methacrylate group.

In an embodiment, the present invention includes methods of fixing a dental carie in a tooth comprising the steps of:
a) drilling the tooth;
b) acid-etching the tooth;
c) administering a dental adhesive wherein the dental adhesive comprises a compound of Formula I

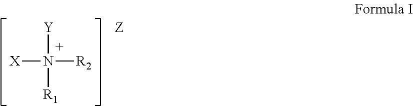

wherein X is selected from the group consisting of —R₄—O—C(O)C(R₅)═CH₂, —CH₂—CH═CH₂, and

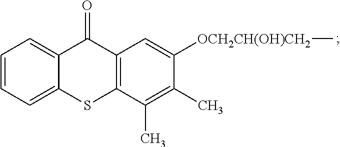

wherein Y is selected from the group consisting of hydrogen, methyl, and —CH₂—CH═CH₂;
or X and Y together with the nitrogen atom to which they are attached form a five membered ring with X and Y having the structure —CH₂—C(R₆)H—C(R₇)H—CH₂—;
wherein Z is a counterion sufficient to balance the charge of the monomer;
wherein R₁ is selected from the group consisting of hydrogen and C₁₋₂alkyl;
wherein R₂ is selected from the group consisting of hydrogen and C₁₋₂alkyl;
wherein R₄ is C₂₋₃alkylene;
wherein R₅ is selected from the group consisting of hydrogen and methyl;
wherein R₆ is an ethylene, acrylate or methacrylate group; and
wherein R₇ is an ethylene, acrylate or methacrylate group.

In certain embodiments, the method may further comprise drilling the tooth and/or acid etching the tooth prior to application of the compound of Formula I. Also, as noted above, each of the embodiments of the methods of administering a compound of Formula I (and/or Formula Ia) for inhibition of MMPs in dental tissue may be used for the method of treating dental caries.

In an embodiment, the present invention includes uses of the compositions disclosed herein such as the compound of Formula I or Ia for the preparation of a medicament for treatment of tooth decay or other appropriate treatment of teeth.

In a variation of the above embodiment, the present invention includes a use of a compound of Formula I for treatment of tooth decay

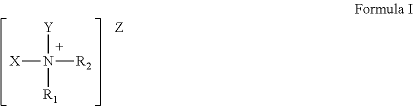

wherein X is selected from the group consisting of —R₄—O—C(O)C(R₅)═CH₂, —CH₂—CH═CH₂, and

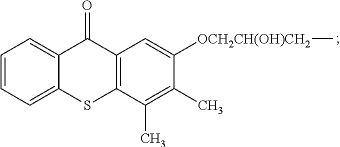

wherein Y is selected from the group consisting of hydrogen, methyl, and —CH₂—CH═CH₂;
or X and Y together with the nitrogen atom to which they are attached form a five membered ring with X and Y having the structure —CH₂—C(R₆)H—C(R₇)H—CH₂—;
wherein Z is a counterion sufficient to balance the charge of the monomer;
wherein R₁ is selected from the group consisting of hydrogen and C₁₋₂alkyl;
wherein R₂ is selected from the group consisting of hydrogen and C₁₋₂alkyl;
wherein R₄ is C₂₋₃alkylene;
wherein R₅ is selected from the group consisting of hydrogen and methyl;
wherein R₆ is an ethylene, acrylate or methacrylate group; and
wherein R₇ is an ethylene, acrylate or methacrylate group;
wherein the compound of Formula I is incorporated into a dental adhesive.

As noted above, each of the embodiments of the compounds of Formula I and Formula Ib and the compositions using these compounds may be employed with the uses for such compounds and/or compositions in the preparation of dental medicaments.

In yet other embodiments, the present invention comprises a method to screen for new compounds that may provide the ability to inhibit MMPs in dentin and/or other dental tissue. The method may comprise the steps of: (a) incubating collagen in the presence of a collagenase and further in the presence and/or the absence of a compound of interest; and (b) assessing the rate at which the collagen degrades in the presence of the compound of interest as compared to in the absence of the compound of interest. In other embodiments, the method may comprise incubating the compound and/or composition of interest with an in vitro model for dentin in the presence of a collagenase and assessing the rate of collagen breakdown. In certain embodiments, the compound of interest may be a compound of Formula I or Formula Ib as disclosed herein. Also, in certain cases, embodiments of the compositions disclosed herein may be included in the formulations to be tested.

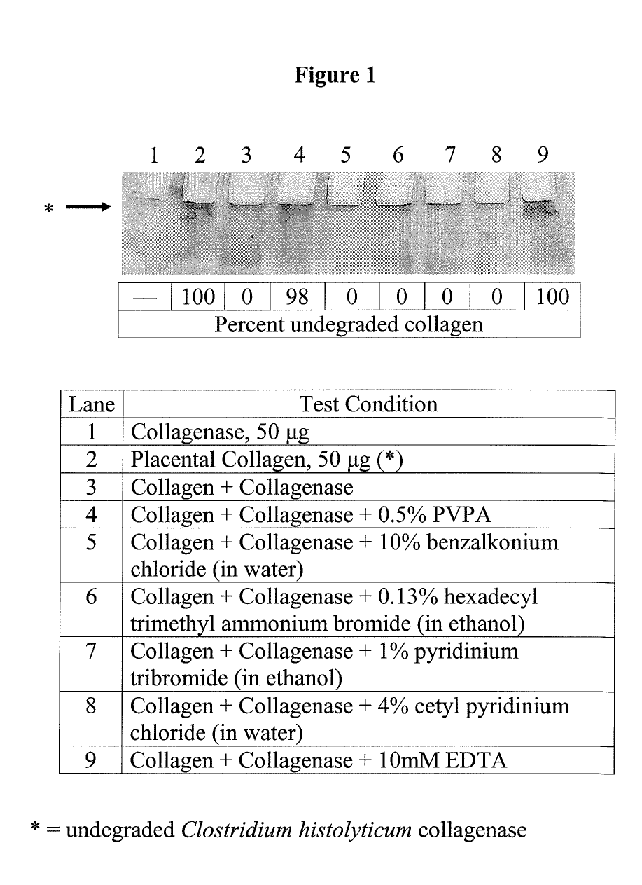

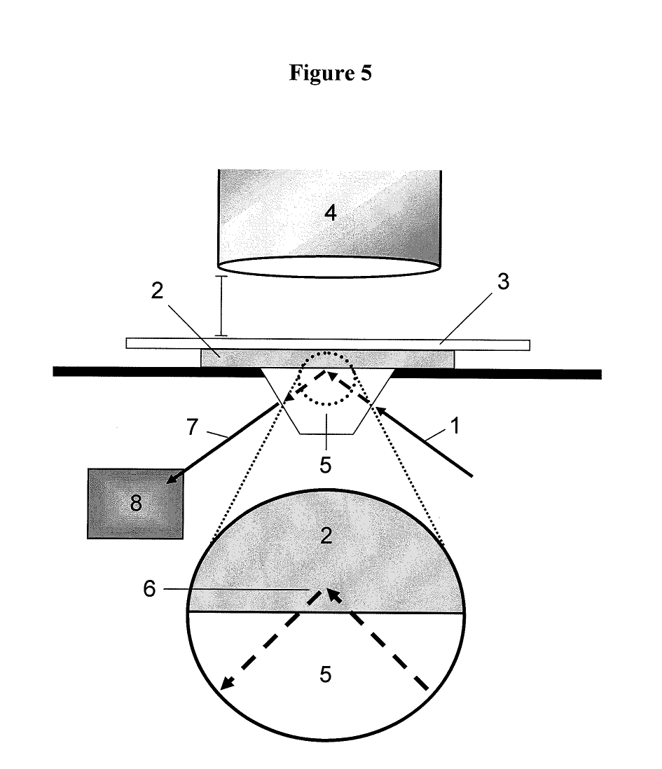

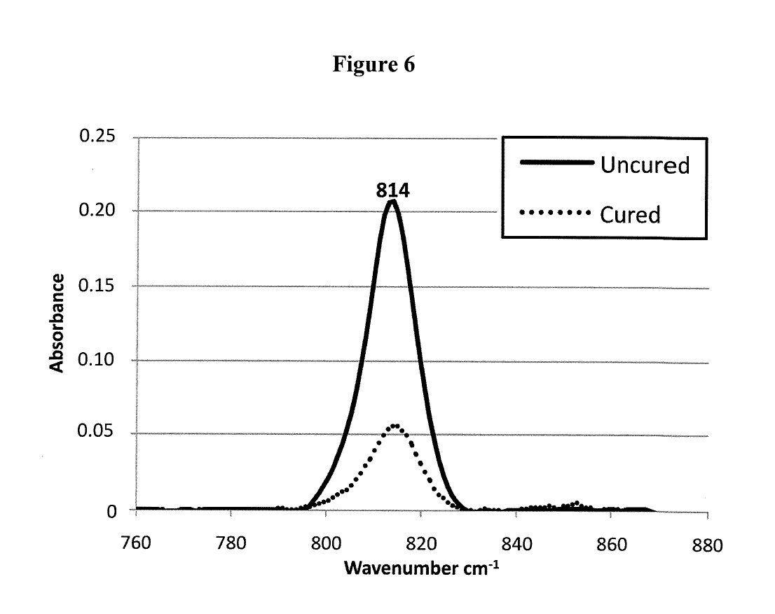

FIGS. 1-4 illustrate some of the various embodiments of methods of the present invention that can be used to identify compounds and/or compositions that may inhibit dentin collagenase activity. Because dentin collagenase is comprised primarily of MMPs, these embodiments effectively demonstrate the inhibition of MMPs. FIGS. 5 and 6 illustrate embodiments of additional compositions of the present invention using diphenyl (2,4,6-trimethylbenzoyl)-phosphine oxide (TPO) as a photopolymerization initiator agent in restorative material compositions containing anti-MMP QACs (quaternary ammonium compounds) and quaternary ammonium methacrylates.

In some embodiments, the collagenase activity and the efficacy of anti-MMP QACs may be assessed by using an in vitro SDS-PAGE assay to quantitate the amount of degradation of type I collagen or type I collagen peptides into their correspondingly smaller peptide fragments. For example, type I collagen or representative type I collagen peptides may be incubated with a collagenase such as, for example, Clostridium histolyticum collagenase or human MMP-9, in the presence or absence of compounds being screened for anti-collagenolytic activity.

In certain embodiments, as discussed in Examples 1-3, reactions may assess the degradation of type I collagen by collagenases, such as, for example, Clostridium histolyticum collagenase. For example, subsequent to incubation, reactions may be electrophoresed on SDS-PAGE gels to determine the amount of undegraded type I collagen present. Undegraded type I collagen can be identified based on its electrophoretic mobility as the protein that is too large to migrate into the separating portion of the gel. Thus, after incubation of the various mixtures and after a voltage is applied to the gel, the undegraded type I collagen remains situated at the junction between the stacking and separating portions of the gel. In some embodiments, quantification may be conducted by using imaging software to quantitate band intensity of undegraded type I collagen for each reaction (one reaction run per well on the gel) relative to degraded type I collagen.

In other embodiments of the present invention, and as discussed in more detail in Examples 1 and 4, reactions may assess the relative amounts of degradation of type I collagen peptides by collagenases such as, for example, MMP-9. In a variation of this embodiment, the type I collagen peptide may be a synthetic peptide or protein that shares amino acid sequence homology with a type I collagen, such as a chromogenic thiopeptolide. The shared sequence identity with the type I collagen may be hydrolytically cleaved by MMP-9 and/other MMPs. In certain embodiments, subsequent to incubation, reactions may be photometrically assessed to determine the amount of undegraded type I collagen peptide present. In some embodiments, degradation of the type I collagen peptide may cause release of a sulfhydryl group (e.g., 2-nitro-5-thiobenzoic acid (TNB)) that may be detected with a color-developing thiol-reactive agent (e.g., 4,4′-dithiodipyridine or Ellman's Reagent) by measuring absorbance at 412 nm.

In alternate embodiments, degradation of type I collagen peptides by collagenases may be assessed using type I collagen peptides labeled with a fluorophor and containing a quencher molecule wherein hydrolytic cleavage of the type I collagen peptide separates the fluorophor from the proximity of the quencher, thereby allowing detection of the fluorophor. In some embodiments, the degradation of the type I collagen peptide may be quantitated by FRET (fluorescence resonance energy transfer) analysis.

To identify the compounds of the present invention that can inhibit collagenase activity, various compounds that were known to possess antimicrobial activity can be assayed to ascertain their ability to inhibit the degradation of collagenase (and consequently be effective anti-MMP compounds). FIG. 1 shows an exemplary embodiment of various antimicrobial agents screened for anti-MMP activity. In particular, FIG. 1 shows a screening analysis of known antimicrobial agents to assess their ability to inhibit type I collagen degradation by Clostridium histolyticum collagenase, wherein only one of the five tested compounds exhibited anti-MMP activity.

After a plurality of screenings by the incubation and gel procedure discussed in Example 1, a variety of quaternary ammonium compounds were identified that possess anti-MMP activity. These compounds were used in subsequent studies as discussed below.

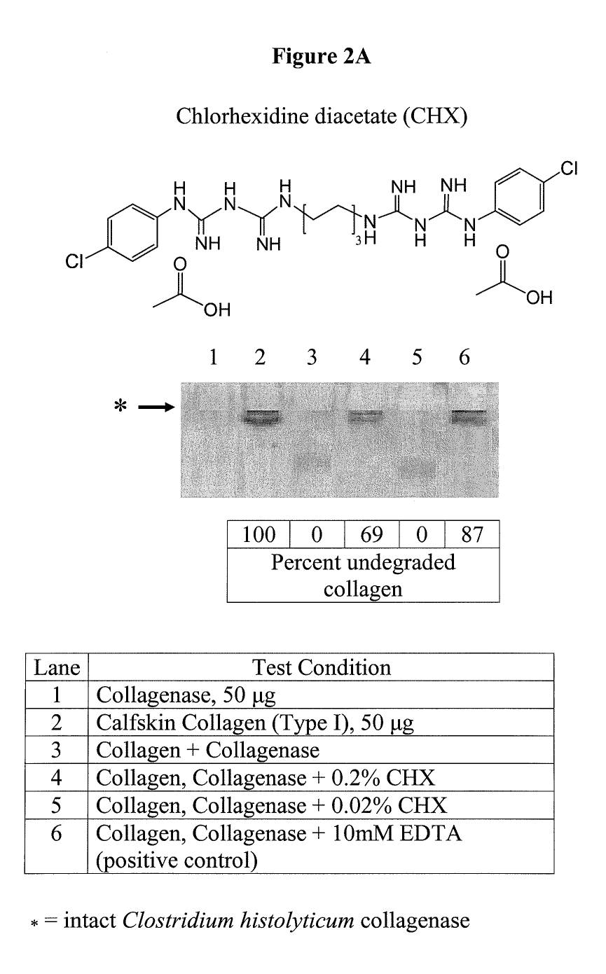

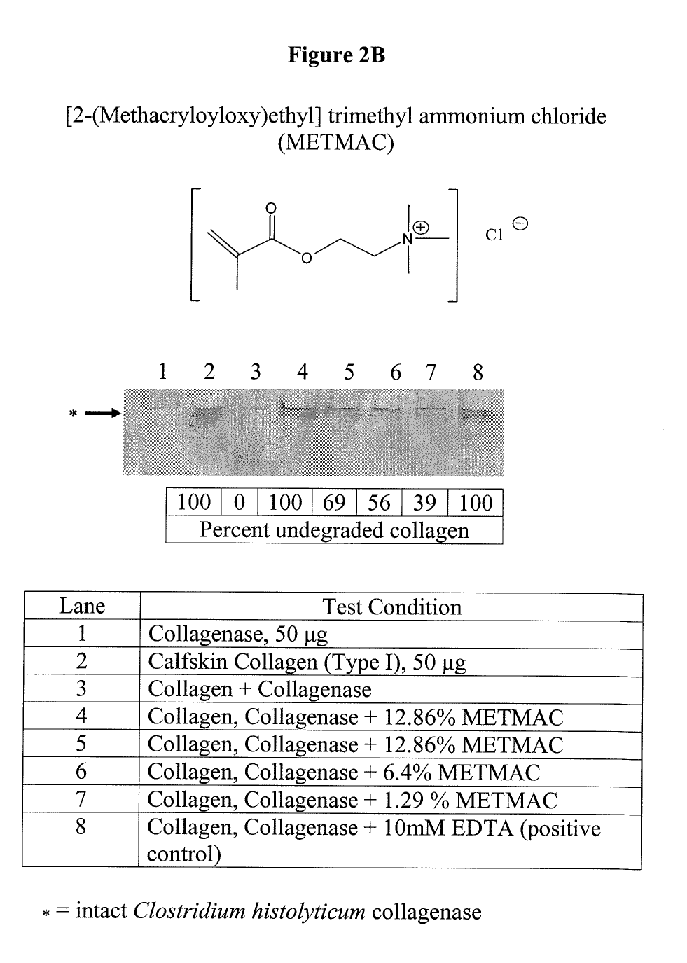

In certain embodiments, compounds identified as having anti-MMP activity and, thus, which may be used in the compositions, methods or uses of the present invention may be tested for concentration dependence in their ability to inhibit MMPs. As exemplified in FIGS. 2A and 2B and summarized in Tables 1 and 2, the concentration of the various compounds may have a variable effect on anti-MMP activity. Table 1 summarizes various compounds tested. Table 2 provides a summary of an analysis of these compounds using an in vitro assay to ascertain their ability to inhibit type I collagen degradation by human MMP-9. Table 2 illustrates that a lower concentration of a compound having anti-MMP activity may correspondingly lower the efficacy of the compound, such as illustrated in FIG. 2A for chlorhexidine. In alternate embodiments, lowering the concentration of a compound having anti-MMP activity may proportionally reduce the efficacy of the compound, such as illustrated by FIG. 2B for [2-(methacryloyloxy)ethyl]trimethylammonium chloride (METMAC).

In yet other embodiments, lowering the concentration of a compound having anti-MMP activity may have little affect on the compound's efficacy, such as illustrated by Tables 1 and 2 for compounds such as 2-acryloxyethyltrimethylammonium chloride (ATA), 2-(methacryloyloxy)ethyltrimethylammonium methyl sulfate (MCMS), diallyldimethylammonium chloride (DDAC) and [3-(methacryloylamino)propyl]trimethylammonium chloride (MAPTAC).

In some embodiments, collagenase activity and the efficacy of anti-MMP QAC methacrylates may be assessed using an in vitro dentin tissue model to quantitate degradation of type I collagen bound within a dentin collagen matrix by collagen-bound endogenous dentin MMPs. For example, demineralized dentin collagen matrix may be incubated in a simulated body fluid in the presence or absence of compounds being screened for anti-collagenolytic activity. In some embodiments, type I collagen degradation is assessed by measuring the amount of dry weight lost from the dentin matrix after incubation, representing loss of type I collagen protein from the dentin collagen matrix. In other embodiments, type I collagen degradation may be assessed by measuring the amount of type I collagen peptides that is released into an incubation solution after incubation. For example, hydrolytic cleavage of type I collagen in the dentin collagen matrix by matrix-bound MMPs releases type I collagen peptide fragments into solution. These type I collagen peptide fragments may be quantitated by treating the incubation solution with an acid to cleave all the peptide bonds, thereby generating the individual constituent amino acids. The amount of hydroxyproline in the incubation solution may be quantitated as a representation of the amount of type I collagen protein content released from the dentin collagen matrix. This provides a relevant assay to ascertain the efficacy of the various anti MMP compounds.

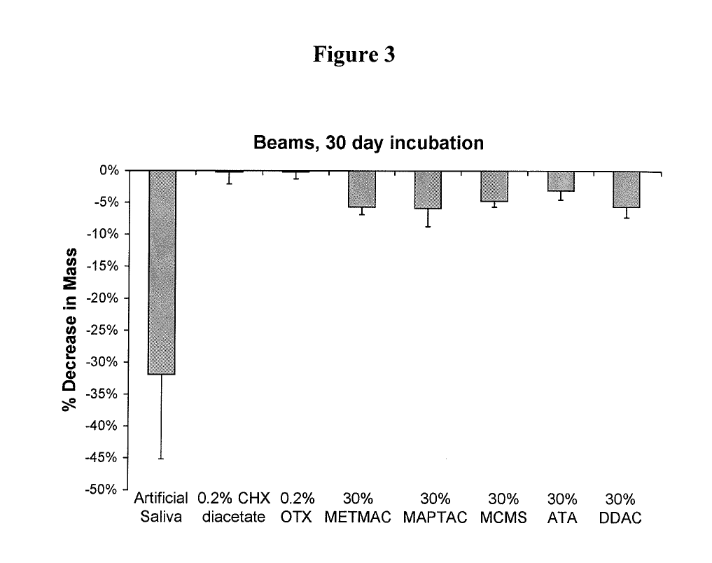

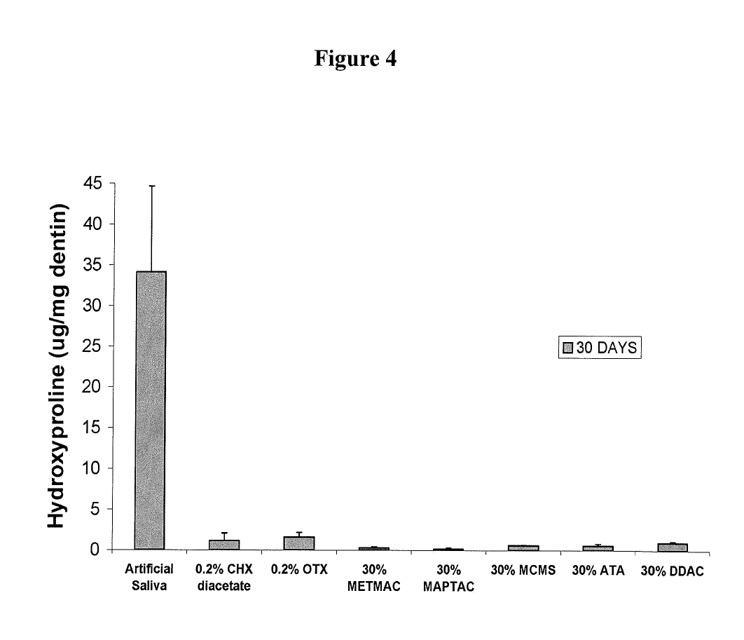

In some embodiments, as illustrated by FIGS. 3 and 4, degradation of type I collagen found in the collagen matrix of demineralized dentin may also be inhibited by QACs of the described invention. For example, demineralized dentin exposed to collagenase in the presence of anti-MMP compounds while in a saliva-like environment loses less mass relative to demineralized dentin exposed to the same conditions save for the absence of any anti-MMP compound. This is due to anticollagenolytic protein degradation as illustrated in FIG. 3. As expected from this result, one correspondingly sees the release of less protein content (i.e., peptide degradation production) into solution as shown in FIG. 4.

In some embodiments, quaternary ammonium methacrylates may be used in the compositions, methods and uses of the invention. These quaternary ammonium methacrylate compositions can be used in restorative materials, such as in adhesives, resins, and cements and mixtures thereof. In certain embodiments, quaternary ammonium methacrylates may be blended with biguanide methacrylates. Other contemporary dental adhesive monomers such as triethylene glycol dimethacrylate (TEGDMA), hydroxyethyl methacrylate (HEMA) or urethane dimethacrylate (UDMA) may also be added. In certain embodiments, dimethacrylates (such as triethylene glycol dimethacrylate) may also be added to provide cross-links between polymer chains to increase resin stiffness and toughness. In certain embodiments, HEMA (hydroxyethylmethacrylate) may be added to facilitate wetting of water-saturated dentin since dimethacrylates (without the corresponding hydrophilic functionalities) are often not very miscible with water.

In some embodiments, the rate of polymerization (rate of cure) or the degree of cure (DC) for the quaternary ammonium restorative materials may be assessed using a diamond-attenuated total reflectance (ATR) element mounted horizontally in an FTIR (Fourier Transform Infrared) spectrometer. For example, as illustrated in FIG. 5, the ATR element may be used to quantitate by FTIR spectrometry the amount of IR light reflected from the restorative material being assessed. In certain embodiments, DC may be measured using the apparatus described in FIG. 5 by quantifying changes in absorbance at the relatively isolated position near 814 cm⁻¹, which represents vinyl group (C═C) values, as illustrated by the exemplary FTIR spectra depicted in FIG. 6. As can be seen in FIG. 6, the disappearance of double bonds from the vinyl, acrylate, or methacrylate monomers upon formation of polymer is shown by a decrease in the signal from the C═C stretch (which occurs at ˜814 cm⁻¹).

As summarized in Table 3, in some embodiments, the use of TPO as a photopolymerization initiator may increase both the rate of cure and the DC for QAC methacrylate resins. For example, the photoinitiation of ATA, MCMS and METMAC QAC methacrylate resins with 0.5 wt % of the commonly used photopolymerization initiator, camphoroquinone (CQ) resulted in DC amounts of 95.9%, 40.5% and 12.6%, respectively. However, when CQ was replaced with 0.1 wt % TPO, the DC for these resins was increased to 100%, 85.8% and 100%, respectively. Similarly, when 1 wt % TPO was added to 30 wt % of ATA, MCMS and METMAC mixed with 40 wt % TEGDMA (tetraethyleneglycol dimethacrylate) and 30 wt % HEMA, the DC at 20 seconds was between 77.6% and 82.1%, as compared to 64.4% DC in the absence of TPO.

In another embodiment, the quaternary ammonium compounds of the present invention may be used in primers for dental restorations to inhibit endogenous MMPs in dentin. Primers allow the dentin's collagen fibers to be “sandwiched” into the resin, resulting in a superior physical and chemical bond of the filling to the tooth. In some embodiments, the primer may comprise (i) a first polymerizable hydrophilic monomer comprising an ethylenic unsaturated group and at least one additional cationic group selected from the group consisting of ammonium bases, pyridinium bases, and phosphonium bases and (ii) a volatile solvent. In certain embodiments, the primer may also comprise (iii) a hydrophilic polymerization photoinitiator. The primer may be applied to acid-etched dentin followed by evaporation of the solvent. In certain embodiments, following application of the primer, the acid-etched dentin may then be covered with an adhesive composition comprising (i) a second polymerizable hydrophilic monomer and (ii) a polymerization photoinitiator. In certain embodiments, the adhesive may be free of volatile solvents.

In yet another embodiment, the quaternary ammonium compounds of the present invention may be used in primer-less adhesive compositions to inhibit endogenous MMPs in dentin, where the components of a primer and components of an adhesive are combined into a single mixture. In some embodiments, the primer-less adhesive may comprise (i) an ethylenic unsaturated group (e.g. acrylic, methacrylic or acrylamide) and at least one cationic group selected from the group consisting of ammonium bases, pyridinium bases and phosphonium bases and (ii) a polymerizable hydrophilic monomer and (iii) a polymerizable hydrophobic monomer, (iv) a polymerization photoinitiator or chemical initiator and (v) a volatile solvent.

In certain embodiments, the primer-less adhesive may be applied to acid-etched dentin in two layers. The first layer serves much like a primer in that it delivers a mixture of a cationic MMP-inhibiting polymerizable monomer, a polymerizable hydrophilic monomer and a polymerizable hydrophobic dimethacrylate monomer in a volatile solvent into the dentin tissue, where it may replace any residual water present with the composition. The second layer serves to deliver more of the same components.

In some embodiments, after evaporating the volatile solvent with a gentle air stream, the primer-less adhesive is polymerized using blue light (ca. 470 nm) from a dental curing unit. The polymerized adhesive would then be covered with a highly-filled resin composite to give the restoration the appearance of tooth structure and better strength and wear resistance after it is photopolymerized.

### EXAMPLES

The invention is described in more detail with reference to the following Examples, which, however are not intended to restrict the scope of the invention. The meanings of some of the abbreviations used hereinabove and in the following Examples are mentioned below along with their chemical formulae.

Abbreviations:
CHX=chlorhexidine diacetate

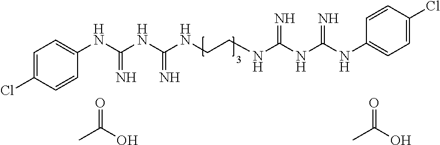

METMAC=[2-(Methacryloyloxy)ethyl]trimethylammonium chloride

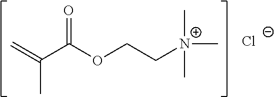

ATA=2-Acryloxyethyltrimethylammonium chloride

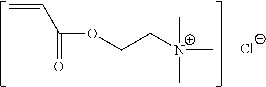

MCMS=2-[(Methacryloyloxy)ethyl]trimethylammonium methyl sulfate

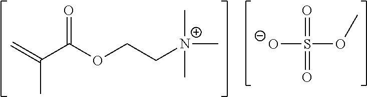

DDAC=Diallyldimethylammonium chloride

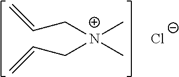

MAPTAC=[3-(Methacryloylamino)propyl]trimethylammonium chloride

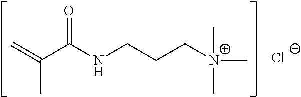

The polymer for MERQUAT 106 has the following repeating unit.

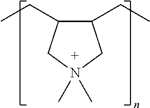

Alternatively, the polymerizable 3 and 4 positions of the pyrrolidine ring may have polymerizable acryl or methacryl groups in the monomer (leading to a slightly different repeating unit from that shown above).
OTX=[3-(3,4-dimethyl-9-oxo-9H-thioxanthen-2-yloxy)-2-hydroxypropyl] trimethyl ammonium chloride

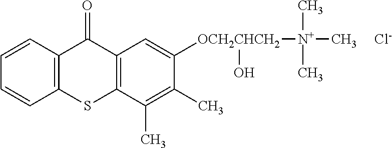

QAC=quaternary ammonium compound(s)
MMP=matrix metalloproteinase

### Example 1

### Assays Used to Screen Compounds of Interest for Anti-MMP Activity

Bacterial collagenase-based anti-MMP screening assay: This assay employs purified Clostridium histolyticum collagenase (hereinafter “collagenase”) as the test enzyme for screening anti-MMP activity of compounds of interest. The screening assay involves incubating a constant concentration of collagenase with Type I soluble collagen and quantifying the amount of Type I collagen that remains undegraded in the presence of collagenase in the presence and absence of different test compounds. The basic assay composition comprises 50 μg of Type I collagen (e.g., human placental; Cat. # C7521; Sigma-Aldrich, St. Louis, Mich., USA) and 50 μg of collagenase (e.g., Clostridium histolyticum E.C. 3.4.24.3; Cat. No. C7667 (1909 CDU/mg solid); Sigma-Aldrich, St. Louis, Mich., USA) in a physiological buffer (pH 7.4) containing zinc ions, with a total reaction volume of 60 μl. This amount of collagenase is sufficient to solubilize 50 μg of Type I soluble collagen in 1 hr at 37° C. in an appropriate incubation buffer (pH 7.4). The buffer composition used comprises 1 M Tris Base, 200 mM NaCl, 10 mM CaCl₂, 0.05 mM ZnCl₂, 3 mM NaN₃ adjusted to pH 7 with 1 N HCl.

Compounds of interest are added to the reaction system described above to assess their ability to inhibit collagenase degradation of collagen. As test compounds vary with respect to molecular weight and affinity for the dentin matrix, a range of concentrations (wt %) for each compound may be added to the basic reaction. Test compounds may be diluted approximately 1:5 in the final reaction volume by the subsequent sequential addition of solutions containing collagenase, collagen, and buffer.

The enzyme and the collagen are kept on ice. Twenty μL of enzyme (concentration of 5 μg protein/μL×20 μL=100 μg of enzyme) protein was incubated with 5 μL of loading buffer and 20 μL of inhibitor. The reaction was started by adding 20 μL of type I collagen (5 μg/μL×20=100 μg collagen). Thus, the total volume was 65 μL. The total protein was 100 μg enzyme protein plus 100 μg of collagen=200 μg protein in 65 μL. Thirty μL of that mixture was applied to the gel (30/65×200 μg protein=92.3 μg protein. Because the total volume was 65 μL and the inhibitor volume was 20 μL, the inhibitor was diluted 3.25-fold.

Following assembly, reactions are incubated for one hour at 37° C. The incubation reactions were terminated by submerging the tube in boiling water for 3 min. After termination, 30 μL of each reaction assay containing 92 μg of total protein is loaded onto a 7.5% SDS-PAGE polyacrylamide gel (BioRad, Hercules, Calif.) and subjected to electrophoresis at 200 V for 60 min. SDS-PAGE gels are routinely run with samples and various controls in the following order. The nature of the controls is further described below. Controls include collagenase alone (lane 1); collagen alone (lane 2); and collagen mixed with collagenase (lane 3). Assay reactions containing collagen mixed with collagenase plus various concentrations of the test collagenase inhibitor compounds are then loaded on the gel starting with lane 4 (loaded in order of decreasing compound concentration). A control is also loaded in the final lane of the gel: 10 mM EDTA, a known, potent anti-MMP compound.

Following electrophoresis of the samples, the gel is stained in 0.25% Coomassie Brilliant Blue overnight and destained for 8 hrs in water prior to imaging. After destaining, the gels are digitally scanned to permit quantification of the amount of undegraded collagen present in each reaction, relative to the collagen standard in lane 2. Using computer software (e.g., Image Tool (University of Texas Health Science Center, San Antonio, Tex., USA), the percent inhibition of collagenase by each concentration of QAC inhibitors can be calculated to allow determination of concentrations that will completely inhibit collagenase.

With regards to the controls, both Type I collagen and collagenase are proteins too large to migrate very far into the SDS-PAGE gel. In particular, Type I collagen can be identified as a strongly stained band at the top of the gel (e.g., lane 2). Because the same amount of Type I collagen is loaded into each of lane 2 (collagen alone), lane 3 (collagen+collagenase), and each of the experimental lanes containing assay samples, lane 2 may be used for quantitative purposes, to represent 100% undegraded Type I collagen and, therefore, 100% inhibition of MMP activity. When collagen and collagenase are incubated together for 1 hr prior to electrophoresis (e.g., lane 3), all of the Type I collagen is degraded into small peptide fragments that migrate into and, subsequently, off, the gel during the 1 hr electrophoresis. As a result, there is no staining of collagen on top of the gel in lane 3. Thus, lane 3 may be used for quantitative purposes to represent 0% undegraded Type I collagen and, therefore, 0% inhibition of MMPs. Lane 3 may also be used to determine the amount of background to be expected in the assay reactions (i.e., where conditions have affected the collagenase used in the reactions such that less than 100% of the Type I collagen in degraded).

Assay reactions containing collagenase mixed with Type I collagen in the presence of a test compound will exhibit varying amounts of undegraded Type I collagen depending on the anti-MMP activity of the test compound. If a test compound completely inhibits collagenase, then no collagen would be destroyed during the 1 hr incubation, and the staining intensity of the collagen at the top of the gel will be as brightly stained as the control collagen in lane 2. If a particular concentration of a QAC is less than 100% effective at inhibiting the collagenase, there will be variable amounts of collagen staining on top of the gels.

Human MMP-9-based anti-MMP screening assay: This assay employed purified human recombinant MMP-9 (Cat. No. 72009) and the Sensolyte Generic MMP colorimetric assay kit (Cat. No. 72095) from AnaSpec, Inc. (San Jose, Calif., USA) for screening anti-MMP activity of compounds of interest. This assay involves incubating a constant concentration of human MMP-9 with a proprietary thiopeptide substrate and quantifying the amount of substrate cleaved by the collagenase in the presence and absence of different test compounds. Cleavage of the proprietary thiopeptide substrate is quantitated by evaluating color production. Reactions may be assembled in a 96-well plate. After incubation for 30 minutes at 37° C., reactions are stopped and absorption measured at 412 nm using a 96-well plate reader synergy HT (BioTek Instruments, Inc., Winooski, Vt., USA).

### Example 2

### Initial Evaluation of Known Antibacterial Compounds Using the Bacterial Collagenase-Based Anti-MMP Screening Assay.

A number of known antibacterial compounds were assayed using the bacterial collagenase-based anti-MMP screening assay described in Example 1. FIG. 1 shows an SDS-PAGE gel upon which the assayed test compounds were run and quantitated from. These experiments revealed that PVPA (0.5 wt %) inhibited collagenase 98%. This is similar to the 10 mM EDTA control lane, which inhibited collagenase 100%. However, benzalkonium chloride (10%), hexadecyl trimethyl ammonium bromide in ethanol (0.15%), pyridinium tribromide (1%) and cetyl pyridinium chloride in water (4%) had no inhibitory activity against collagenase.

### Example 3

### Quantitative Evaluation of Compounds Using the Bacterial Collagenase-Based Anti-MMP Screening Assay.

Additional test compounds were screened using the bacterial collagenase-based anti-MMP screening assay described in Example 1. For example, see FIG. 2, Panels A and B, showing exemplary SDS-PAGE electrophoresis gels upon which samples were run to assess the anticollagenolytic activity of CHX and METMA. The compounds tested and the results of these experiments are summarized in Table 1.

FIG. 2A shows the inhibitory activity of 0.02 and 0.2% chlorhexidine diacetate (CHX), a biguanide agent on Clostridium histolyticum collagenase. At a concentration of 0.02%, CHX did not inhibit collagenase (lane 5). However, at a concentration of 0.2%, CHX inhibited collagenase 69%. In lane 6, 10 mM EDTA inhibited collagenase 87%.

FIG. 2B shows the inhibitory activity of [2-(methacryloxy)ethyl] trimethyl ammonium chloride (METMAC), a polymerizable quaternary ammonium derivative of methacrylate. In lane 4, the mixture of collagen and collagenase was incubated together with 25.7 wt % METMAC to see if it could inhibit the collagenase. After 1 hr of electrophoresis, the collagen remained on the origin and stained as blue as did the collagen control in lane 2. That is, 25.7 wt % METMAC inhibited the Clostridium collagenase 100% (lane 4). In lane 5, the inhibitory activity of 12.85% METMAC was tested. It inhibited collagenase 69%. When the METMAC concentration was reduced to 6.4%, it inhibited collagenase 56% (lane 6). When the METMAC concentration was reduced to 1.3%, it inhibited collagenase 39%. Thus, METMAC is an excellent inhibitor of collagenase. The positive control, 10 mM EDTA inhibited collagenase 100%. To the inventors' knowledge, this is the first direct evidence that a polymerizable quaternary ammonium methacrylate monomer can inhibit collagenase.

While results indicated that some of compounds show a correlation between amount of compound used in the assay and the amount of MMP inhibition observed, others did not. For example, 2-acryloxyethyltrimethyl ammonium chloride (ATA) resulted in 73% inhibition of collagen at 16 wt %. Reducing the ATA concentration to 8%, 4%, 1% and 0.5% inhibited collagenase 49%, 44%, 55% and 57%, respectively. Other compounds that behaved similarly are MCMS and MAPTAC. Without being bound by any theory, the inventors believe that a lack of a proportional dose-response observed in the described experiments may reflect that ATA interacts with collagen in such a manner as to produce elevated concentrations of the compound on or in very close proximity to collagen as compared to the overall concentration of ATA in the solution of the reaction tube environment. That is, ATA may have a partition coefficient greater than 1.0.

Table 1, found below, summarizes the above described experimental results.

TABLE 1
Inhibition of Clostridium histolyticum
collagenase (SDS-PAGE gel analysis)

| | | COLLAGENASE |
|---|---|---|
| INHIBITOR | WT % | INHIBITION (%) |
| Chlorhexidine diacetate | 0.2% | 74% |
| (in water) | 0.02% | 1% |
| METMAC | 25.7% | 100% |
| | 12.9% | 69% |
| | 6.4% | 56% |
| | 1.3% | 39% |
| ATA | 16% | 73% |
| | 8% | 49% |
| | 4% | 44% |
| | 1% | 55% |
| | 0.5% | 57% |
| MCMS | 16% | 87% |
| | 8% | 62% |
| | 4% | 65% |
| | 1% | 70% |
| | 0.5% | 73% |
| DDAC | 12% | 73% |
| | 6% | 66% |
| | 3% | 59% |
| | 1% | 56% |
| | 0.5% | 50% |
| MAPTAC | 10% | 55% |
| | 5% | 53% |
| | 1% | 53% |
| | 0.5% | 39% |
| | 0.1% | 41% |
| OTX | 0.1% | 57% |
| | 0.2% | 86% |

### Example 4

### Quantitative Evaluation of Compounds Using the Human MMP-9-Based Anti-MMP Screening Assay.

The test inhibitors screened in the experiments described in Example 3 were also screened using the human MMP-9-based anti-MMP screening assay described in Example 1. Note, OTX ([3-(3,4-dimethyl-9-oxo-9H-thioxanthen-2-yloxy)-2-hydroxypropyl] trimethyl ammonium chloride) was not tested using this assay due to its intrinsic yellow coloration even at low concentrations (e.g., 0.5%), which interfered with the colorimetric assay output.

ATA (2-acryloxyethyltrimethyl ammonium chloride at a concentration of 24 wt % inhibited soluble human MMP-9 82% (Table 2). When it was diluted to 10 wt %, it still inhibited the enzyme 79% indicating that this inhibitor is very potent. The methacrylate derivative of that inhibitor (METMAC) when used at 24 or 10 wt % also inhibited MMP-9 by 71% and 42%, respectively. MCMS (2-[(methacryloyloxy)ethyl[trimethylammonium methyl sulfate) at 24 and 10 wt %, inhibited MMP-9 84 and 55%, respectively. DDAC (diallyldimethylammonium chloride), when used at 24 and 10 wt % only inhibited MMP-9 by 16 and 7%, respectively. When MAPTAC [3-(methacryloylamino)propyl] trimethyl ammonium chloride was screened at 24 and 10 wt %, respectively, which resulted in inhibition of MMP-9 by 17 and 6%, respectively. The last compound tested in Table 2 is a cationic polymer called MERQUAT 106 (Nalco Company (Naperville, Ill., USA)). It inhibited MMP-9 levels 100% (20% MERQUAT) and 63% (10% MERQUAT).

TABLE 2
Inhibition of soluble human MMP-9
by quaternary ammonium compounds

| | % MMP ACTIVITY INHIBITION |
|---|---|
| INHIBITOR | (t = 30 minutes) |
| 24% ATA | 82% |
| 10% ATA | 79% |
| 24% MCMS | 84% |
| 10% MCMS | 55% |
| 24% METMAC | 71% |
| 10% METMAC | 42% |
| 24% DDAC | 16% |
| 10% DDAC | 7% |
| 24% MAPTAC | 17% |
| 10% MAPTAC | 6% |
| 20% MERQUAT | 100% |
| 10% MERQUAT | 63% |

### Example 5

### Evaluation of Anti-MMP Activity Using Collagen Matrix Based Assays

Clinically, endogenous MMPs in dentin are not soluble, but rather are bound to collagen. Using an in vitro model system (Carrilho et al., 2009), mid-coronal dentin disks 1 mm thick were prepared from extracted, unerupted human third molars. Beams 2×1×7 mm were cut from the disks and completely demineralized in 10% phosphoric acid for 12 hr at 25° C. This procedure uncovers all the collagen and their bound noncollagenous proteins, including MMPs, and activates these proteases.

In this experiment, we assessed the anti-collagenolytic activity of the following compounds: OTX (0.2 wt %), METMAC (30%), MAPTAC (30 wt %), MCMS (30 wt %), ATA (30 wt %) and DDAC (30 wt %). Chlorhexadine (0.2 wt %) was used as a positive control.

In control beams incubated in artificial saliva (AS) containing buffer (pH 7), 10 mM calcium, and 0.05 mM zinc, the beams lost over 30% of their dry mass in 30 days (37° C.) (FIG. 3). Experimental beams incubated in AS containing effective inhibitory concentrations of biguanides or QACs, lost much less dry mass (FIG. 3).

We collected the 1 mL of artificial saliva (AS) used as the incubation medium for the demineralized dentin beams in the above-described experiment and hydrolyzed it in HCl to reduce any solubilized collagen to its constituent amino acids. Since hydroxyproline is only found in collagen, by analyzing the hydroxyproline content of the medium, we could calculate the amount of collagen that solubilized from the insoluble dentin matrix over 30 days of incubation. FIG. 4 shows that specimens incubated in artificial saliva solubilized 35 μg of hydroxyproline/mg dry weight of dentin matrix. When the artificial saliva contained MMP-inhibitors, less than 1 μg of hydroxyproline/mg dry weight of matrix was detected. This indicates that these MMP-inhibitors prevented solubilization of collagen peptide fragments over the 30 day incubation period. The quaternary ammonium compound OTX is not only an excellent MMP-inhibitor, it can be used as a hydrophilic photosensitizer (Ye et al., 2009). That is, the effective concentration of OTX as an MMP-inhibitor and as a photosensitizer are the same.

### Example 6

### Materials and Methods

Resin composition: Three quaternary ammonium methacrylates available as aqueous solutions were selected for the study: 1) 2-acryloxyethyltrimethyl ammonium chloride (ATA) (Cat. #496146; Sigma-Aldrich, St. Louis, Mich., USA) (80 wt % monomer in 20 wt % water); 2) 2-[methacryloyloxy)ethyl]trimethylammonium methyl sulfate (MCMS) (Cat. #408123; Sigma-Aldrich, St. Louis, Mich., USA) (80 wt % monomer in 20 wt % water); 3) [2-(methacryloyloxy)ethyl]trimethylammonium chloride (METMAC) (Cat. #408107; Sigma-Aldrich, St. Louis, Mich., USA) (75 wt % monomer in 25 wt % water). All quaternary ammonium methacrylates were made photocurable by inclusion of 1 wt % 2-ethyl-4-dimethylaminobenzoate (EDMAB) and 0.5 wt % camphoroquinone (CQ) or 1 wt % TPO (diphenyl(2,4,6-trimethylbenzoyl)-phosphine oxide) alone (Table 3) and homopolymerized by conventional free radical polymerization techniques. In addition, comonomer blends containing 30 wt % of each quaternary ammonium methacrylate solution (ATA or MCMS or METMAC), 40% triethyleneglycol dimethacrylate (TEGDMA) and 30% 2-hydroxyethyl methacrylate (HEMA) were prepared. These blends were made photocurable by inclusion of 1 wt % TPO (Table 3). A 40 wt % TEGDMA and 60 wt % HEMA mixture with 1 wt % TPO was used as a control. Table 3 summarizes the DC of quaternary ammonium methacrylates and their TEGDMA/HEMA blends.

Measurement of monomer conversion (DC): To measure monomer conversion, Fourier transform infrared (FTIR) spectroscopy was conducted with an attenuated total reflection (ATR) accessory unit (Golden Gate ATR, MkII, SPECAC Inc., Cranston, R.I., USA) mounted on an FTIR spectrometer (FTS-40, Digilab/BioRad, Cambridge, Mass., USA). A schematic illustrating the ATR-FTIR spectrometer and its use is depicted in FIG. 5. The ATR accessory unit operates by measuring the changes that occur in a internally reflected infrared beam existing entirely within the ATR accessory when the IR beam (1) comes into contact with a sample (2). One drop of each experimental QAC methacrylate or resin mixture was placed on the diamond crystal (2) of the TCS-ATR (heated to 30° C.) using a 1 mL disposable syringe (Norm-Ject, Tuttlingen, Germany). A 1.5×1.5 cm×76 μm piece of polyester film (3) (Mylar, Type D, Polymer Plastics Corporation, Reno, N.Y., USA) was immediately placed over the top of the deposited resin sample (2) to exclude oxygen. A quartz-tungsten-halogen light-curing unit (4) (Optilux 400, Demetron/Kerr, Danbury, Conn., USA) with an output intensity of 525 mW/cm² as measured using a radiometer (Optilux Radiometer, Demetron/Kerr, Danbury, Conn., USA) was used to photopolymerize the resin samples (2) through the polyester strip for 20 sec, 40 sec, or 60 sec exposures at a tip distance of 2 mm. The experimental setup simulated dispensing of an adhesive resin in a thin layer on a tooth prepared surface clinically, but without air-drying.

An infrared beam (1) is directed onto an optically dense crystal (5) with a high refractive index at a certain angle. Here, because the samples (2) are thin films of solidified resin made using experimental resin composites, a diamond crystal (5) is used. The diamond crystal (5) is generally a parallel-sided horizontal plate, typically of about 2 mm thickness, with the upper surface exposed. The small area diamond crystal (5) generally provides only a single reflection. The internal reflectance of the IR beam (1) creates an evanescent wave (6) within the diamond crystal (5) that extends beyond the surface of the diamond crystal (5) into the sample (2). The evanescent wave (6) extends only a few microns (0.5μ-5μ) beyond the diamond crystal surface (5) and into the sample (2). In regions of the infrared spectrum where the sample (2) absorbs energy, the evanescent wave (6) will be attenuated or altered. The attenuated energy from each evanescent wave is passed back to the IR beam (7), which then exits the opposite end of the diamond crystal (2) and is passed to the detector (8) in the IR spectrometer. The system then generates an infrared spectrum. For the technique to be successful, the sample (2) generally should be in direct contact with the diamond crystal (5), and the refractive index of the diamond crystal (5) generally should be significantly greater than that of the sample (2) or else internal reflectance will not occur (rather, the light will be transmitted instead of internally reflected in the diamond crystal (5)).

Kinetic infrared (IR) spectra were obtained between 4000 and 700 cm⁻¹ at 2 cm⁻¹ resolution. Spectral acquisition was initiated immediately upon resin droplet deposition to obtain the IR spectra of each solution group in the uncured state. The curing light was activated 5 s after droplet deposition. After the photo-curing exposure, any post-cure polymerization was allowed to continue up to 120 s from light initiation. Monomer conversion was determined using changes in the relatively isolated absorbance near 814 cm⁻¹, representing vinyl C═C values (Wiles, K. B., et al., J. Polymer. Sci., 2004, 42: 2294-3001) (FIG. 6). Absorption values on either side of the peak of interest were baseline corrected to zero, and the peak height was determined using a baseline connecting absorbance tails on either side of the peak (FIG. 6). Because there were no overlapping peaks at that wave number, the degree of conversion was calculated as the linear decrease in peak height relative to height of the peak in the unexposed state using the baseline as zero. Five repetitions for each test condition were made.

The rate of cure was obtained by calculating the derivative of the smoothed conversion curve, intended as the trendline fitting the conversion vs. time curve, using data-analysis software (Logger Pro 3.5, Vernier Software & Technology, Beaverton, Oreg.). The rate of cure is how fast the mixture polymerizes. The time of cure means the number of seconds to reach the maximum degree of cure. Dentists do not like mixtures that take 20-30 sec to reach full cure. The maximum polymerization rate (expressed as DC (%/s)) was obtained from the degree of conversion vs. time curves in the first 20 s exposure.

Statistical analysis: A three-way ANOVA was used to evaluate how the main factors of resin type, initiator and exposure time affected both the rate and extent of the DC. Differences between groups were calculated using Tukey's post hoc test. A two-way ANOVA was used to evaluate how the main factors of resin type and exposure time affected both the rate and extent of the DC of QACs polymerized with TPO compared to resin mixtures and control. Differences between groups were calculated using Tukey's post hoc test. Statistical significance was preset at α=0.05.

Results

The efficacy of CQ/EDMAB and TPO photoinitiators on the conversion of a number of quaternary ammonium methacrylates alone or blended with typical dental adhesive monomers was evaluated and compared using FTIR spectroscopy. FIG. 5 illustrates the FTIR spectroscopy method used for the experiments described below. The results of these experiments are summarized in Table 3.

Three quaternary ammonium methacrylates were evaluated: ATA, MCMS and METMAC. DDAC and MAPTAC were not evaluated because they did not inhibit Clostridium collagenase (Table 1) or MMP-9 (Table 2) as effectively as ATA, MCMS and METMAC.

When ATA, MCMS and METMAC were photoinitiated with 0.5 wt % CQ and 1% EDMAB for 20 sec (Table 3), although the degree of conversion (DC) of ATA was 95.9%, it was only 40.5% for METMAC and 12.6% for MCMS.

In contrast, when these same quaternary ammonium methacrylates were photoinitiated with 1 wt % TPO for 20 sec, the DC of ATA and METMAC were 100% while that of MCMS was 85.8%. Thus, TPO increased both the rates of cure and the DC. We interpret this superior performance of TPO as being due to its water solubility. All three quaternary methacrylates contained 20-25% water. Based on these results, TPO was selected as the photoinitator for polymerizing ATA, MCMS and METMAC when blended with TEGDMA/HEMA.

When 30 wt % of ATA, MCMS and METMAC were mixed with 40 wt % TEGDMA, a dimethacrylate, and 30 wt % HEMA using 1 wt % TPO as a photoinitiator, the DC after 20 s were between 77.6 and 82.1%, while the DC of the 40% TEGDMA/60% HEMA was only 64.4%. Thus, addition quaternary ammonium methacrylates significantly (p<0.05) increased the DC of the adhesive blends at 20, 40 or 60 sec. The rate of cure of ATA, MCMS and METMAC blends was significantly lower than the neat resins because TEGDMA, being that dimethacrylate facilitates the gel effect that limits diffusion of reactants resulting in autodeceleration of conversion rate and limiting final conversion (Dickens et al., 2003).

The time to reach maximum DC when using TPO as a photoinitiator was only 5.0-6.4 sec for the neat resins (i.e., anhydrous) and 9.8-11.2 sec for the blends. These times to reach maximum DC are even lower than many marketed adhesives, making their use very desirable.

Model resin blends containing TEGDMA and HEMA were selected to simulate dental adhesive systems. Presumably, the incorporation of QAC methacrylates to dimethacrylate resin blends increased DC because it lowered the viscosity of the comonomer blends to the extent that it increased polymer chain mobility and increased the rate of diffusion of radicals.

In addition to the photoinitiator employed, the duration of light exposure is important in optimizing the performance of adhesive resins. Manufacturers usually recommend only 10-20 s of light exposure with halogen-based light-curing unit for dental adhesive systems. The results of the above-described experiments indicate that this length of time is insufficient for both QACs and QAC-based dimethacrylate blends, and confirmed that prolonged light exposure contributes to reducing the percentage of uncured monomers of adhesive resins (Cadenaro, M., et al., Eur. J. Oral Sci., 2005, 113: 525-530). These results suggest that the degree of cure of QAC methacrylates is affected by the photo-initiating system. The water-compatible photo-initiator TPO increased the DC of QAC methacrylate aqueous solutions compared to CQ/EDMAB. The incorporation of QAC methacrylates in TEGDMA-HEMA resin blends increased the DC of the adhesive resin mixtures.

TABLE 3
Degree of cure (DC) at different light exposure durations, maximum rate of cure (DC %/s) and
time of exposure to maximum rate of cure.

| | | | | | | Rate of | |
|---|---|---|---|---|---|---|---|
| | | | DC % | DC % | DC % | cure | Time |
| QAM | Dimethacrylates | Initiator | 20 s | 40 s | 60 s | (%/sec) | (s) |
| ATA | None | 0.5 wt % CQ + | 95.9 ± 0.4^{IJ} | 99.4 ± 0.2^{JKL} | 100.1 ± 0.3^{KL} | 13.9 ± 1.3^{D} | 6.2 ± 0.4 |
| 20% H₂O | | 1 wt % EDMAB | | | | | |
| MCMS | None | 0.5 wt % CQ + | 12.6 ± 2.8^{A} | 22.4 ± 3.1^{B} | 29.1 ± 2.8^{C} | 1.0 ± 0.1^{A} | 10.4 ± 1.4 |
| 20% H₂O | | 1 wt % EDMAB | | | | | |
| METMAC | None | 0.5 wt % CQ + | 40.5 ± 1.8^{D} | 64.1 ± 2.3^{E} | 75.2 ± 2.4^{F} | 2.3 ± 0.1^{B} | 7.0 ± 0.6 |
| 25% H₂O | | 1 wt % EDMAB | | | | | |
| ATA | None | 1 wt % TPO | 102.1 ± 0.6^{Lj} | 101.3 ± 0.6^{Lj} | 101.7 ± 0.71^{Lj} | 35.4 ± 0.9^{Fe} | 5.0 ± 0.6 |
| 20%H₂O | | | | | | | |
| MCMS | None | 1 wt % TPO | 85.8 ± 0.7^{Gfg} | 94.1 ± 0.9^{Hi} | 96.6 ± 1.7^{IJKi} | 9.2 ± 0.4^{Cc} | 6.2 ± 1.0 |
| 20% H₂O | | | | | | | |
| METMAC | None | 1 wt % TPO | 100.9 ± 0.6^{Lj} | 101.1 ± 0.8^{Lj} | 101.7 ± 0.71^{Lj} | 15.5 ± 0.5^{Ed} | 6.4 ± 0.5 |
| 25% H₂O | | | | | | | |
| 30 wt % | 40 wt % | 1 wt % TPO | 80.1 ± 2.4^{cd} | 84.2 ± 3.1^{efg} | 85.6 ± 2.9^{fg} | 9.9 ± 0.7^{c} | 9.8 ± 0.4 |
| ATA | TEGDMA | | | | | | |
| | 30 wt % HEMA | | | | | | |
| 30 wt % | 40 wt % | 1 wt % TPO | 82.1 ± 0.4^{de} | 85.9 ± 0.3^{fg} | 87.3 ± 0.7^{h} | 9.5 ± 0.1^{c} | 11.2 ± 0.4 |
| MCMS | TEGDMA | | | | | | |
| | 30 wt % HEMA | | | | | | |
| 30 wt % | 40 wt % | 1 wt % TPO | 77.6 ± 0.3^{c} | 81.3 ± 0.5^{de} | 82.9 ± 0.4^{def} | 8.7 ± 0.3^{bc} | 8.2 ± 0.4 |
| METMAC | TEGDMA | | | | | | |
| | 30 wt % HEMA | | | | | | |
| None | 40 wt % | 1 wt % TPO | 64.4 ± 0.9^{a} | 70.3 ± 0.9^{b} | 72.2 ± 1.0^{b} | 6.6 ± 0.3^{a} | 17.6 ± 0.5 |
| | TEGDMA | | | | | | |
| | 60 wt % HEMA | | | | | | |

Abbreviations:
QAM = quaternary ammonium methacrylate
ATA = 80 wt % 2-acryloxyethyltrimethyl ammonium chloride + 20 wt % water
MCMS = 80 wt % 2-[methacryloyloxy)ethyl]trimethylammonium methyl sulfate + 20 wt % water
METMAC = 75 wt % [2-(methacryloyloxy)ethyl]trimethylammonium chloride + 25 wt % water
TEGDMA = triethyleneglycol dimethacrylate;
HEMA = 2-hydroxyethyl methacrylate
CQ = camphoroquinone;
EDMAB = ethyl-4-dimethylaminobenzoate
TPO = diphenyl(2,4,6-trimethylbenzoyl)-phosphine oxide.

Values are mean±SD (n−5). Means followed by the same capital superscript letter indicate no difference (p<0.05) between initiators. Means followed by the same lower-case superscript letter indicate no difference (p<0.05) among TPO-added resins.

The present invention has been described generally and with an emphasis on particular embodiments. It should be apparent to those of ordinary skill in the art that modifications can be made to the above disclosure and still fit within the scope and spirit of the invention. It is intended, contemplated, and therefore within the scope of the invention to combine any of the plurality of different elements in each of the embodiments in the above disclosure with any other embodiment. Moreover, when a range is disclosed, any number that falls within the range is a contemplated end point, even if that number is not explicitly disclosed. Also, although certain exemplary embodiments and methods have been described in some detail, for clarity of understanding and by way of example, it will be apparent from the foregoing disclosure to those skilled in the art that variations, modifications, changes and adaptations of such embodiments and methods may be made without departing from the true spirit and scope of the invention. Where methods and steps described above indicate certain events occurring in certain order, those of ordinary skill in the art will recognize that the ordering of certain steps may be modified and that such modifications are in accordance with the variations of the invention. Furthermore, certain steps may be performed concurrently in a parallel process when possible, as well as performed sequentially as described above. Therefore, the above description should not be taken as limiting the scope of the invention but rather the invention should be defined by the below claims. Moreover, the list of any references mentioned in the disclosure is herein incorporated by reference in their entirety.

That which is claimed is:

1. A composition comprising polymerizable quaternary ammonium monomers capable of inhibiting matrix metalloproteinase activity in dental tissue.

2. The composition of claim 1, wherein the composition is a dental adhesive.

3. The composition of claim 1, wherein the polymerizable quaternary ammonium monomers are compounds of Formula I 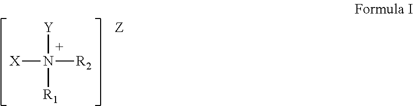
wherein X is selected from the group consisting of —R₄—O—C(O)C(R₅)═CH₂, —CH₂—CH═CH₂, and 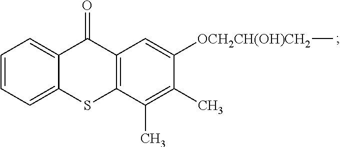
wherein Y is selected from the group consisting of hydrogen, methyl, and —CH₂—CH═CH₂;
or X and Y together with the nitrogen atom to which they are attached form a five membered ring with X and Y having the structure —CH₂—C(R₆)H—C(R₇)H—CH₂—;
wherein Z is a counterion sufficient to balance the charge of the monomer;
wherein R₁ is selected from the group consisting of hydrogen and C₁₋₂alkyl;
wherein R₂ is selected from the group consisting of hydrogen and C₁₋₂alkyl;
wherein R₄ is C₂₋₃alkylene;
wherein R₅ is selected from the group consisting of hydrogen and methyl;
wherein R₆ is an ethylene, acrylate or methacrylate group; and
wherein R₇ is an ethylene, acrylate or methacrylate group.

4. The composition of claim 3, wherein the polymerizable quaternary ammonium monomers are compounds of Formula Ia 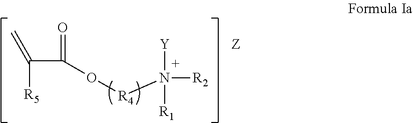
wherein R₄ is ethylene and wherein R₅ is a methyl group.

5. The composition of claim 3, wherein the compounds are selected from the group consisting of [2-(methyl-acryloyloxy)ethyl-N-trimethyl ammonium chloride (METMAC), 2-(methacryloyloxy)ethyltrimethylammonium methyl sulfate (MCMS), 2-Acryloxyethyltrimethylammonium chloride (ATA), diayllyldimethyl ammonium chloride (DDAC), 3-[3,4-dimethyl-9-oxo-9H-trioxanthen-2-yloxy]-2-hydroxypropyl] trimethyl ammonium chloride (QTX), [3-(methacryloylamino) propyl]trimethylammonium chloride (MERQUAT 106), and N—N-dimethylaminomethacrylate methyl chloride.

6. The composition of claim 5 further comprising chlorhexidine (CHX).

7. The composition of claim 3 further comprising diphenyl (2,4,6-trimethylbenzoyl)-phosphine oxide (TPO) and/or benzoyl peroxide.

8. The composition of claim 3, wherein
wherein Y is selected from the group consisting of methyl, and —CH₂—CH═CH₂;
wherein Z is a counterion selected from the group consisting of a halo group, a methyl sulfate group, or an acetate group;
wherein R₁ is methyl;
wherein R₂ is methyl;
wherein R₄ is ethylene; and
wherein R₅ is selected from the group consisting of hydrogen and methyl.

9. The composition of claim 1, wherein the polymerizable quaternary ammonium monomers are present in the composition in concentrations from about 0.2 to 40% by weight.

10. A method of inhibiting matrix metalloproteinase activity in dental tissue in a subject comprising administering to the subject a dental composition comprising polymerizable quaternary ammonium monomers of Formula I 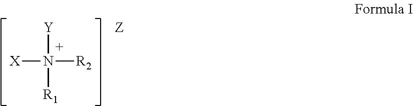
wherein X is selected from the group consisting of —R₄—O—C(O)C(R₅)═CH₂, —CH₂—CH═CH₂, and 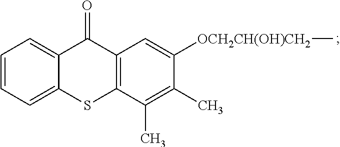
wherein Y is selected from the group consisting of hydrogen, methyl, and —CH₂—CH═CH₂;
or X and Y together with the nitrogen atom to which they are attached form a five membered ring with X and Y having the structure —CH₂—C(R₆)H—C(R₇)H—CH₂—;
wherein Z is a counterion sufficient to balance the charge of the monomer;
wherein R₁ is selected from the group consisting of hydrogen and C₁₋₂alkyl;
wherein R₂ is selected from the group consisting of hydrogen and C₁₋₂alkyl;
wherein R₄ is C₂₋₃alkylene;
wherein R₅ is selected from the group consisting of hydrogen and methyl;
wherein R₆ is an ethylene, acrylate or methacrylate group; and
wherein R₇ is an ethylene, acrylate or methacrylate group.

11. The method of claim 10, wherein the dental composition comprises an acid-etching solution, a primer, an adhesive or a cement, or mixtures thereof.

12. The method of claim 10, wherein the polymerizable quaternary ammonium monomers is a compound of Formula Ia: 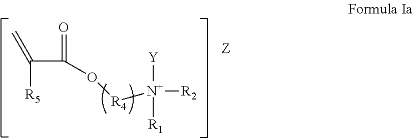
wherein R₄ is ethylene and wherein R₅ is a methyl group.

13. The method of claim 10, wherein
Y is selected from the group consisting of methyl, and —CH₂—CH═CH₂;
wherein Z is a counterion selected from the group consisting of a halo group, a methyl sulfate group, or an acetate group;
wherein R₁ is methyl;
wherein R₂ is methyl;
wherein R₄ is ethylene; and
wherein R₅ is selected from the group consisting of hydrogen and methyl.

14. The method of claim 10, wherein the polymerizable quaternary ammonium monomers are present in the composition in concentrations from about 0.2 to 40% by weight.

15. The method of claim 11, wherein the dental composition further comprises a photopolymerizable initiator and one or more of the following: (i) a polymerizable monomer containing an acid group, (ii) a polymerizable monomer selected from the group consisting of pyridinium bases and phosphonium bases, (iii) a hydrophilic polymerizable monomer, (iv) a hydrophobic polymerizable monomer, and (v) a polymerizable dimethacrylate monomer.

16. The method of claim 15, wherein the photopolymerizable initiator is TPO.

17. The method of claim 16, wherein the dental composition comprises a member from each of (i) a polymerizable monomer containing an acid group, (ii) a polymerizable monomer selected from the group consisting of pyridinium bases and phosphonium bases, (iii) a hydrophilic polymerizable monomer, and (iv) a polymerizable dimethacrylate monomer.

18. The method of claim 15, wherein the dental composition further comprises a photopolymerization inhibitor.

19. A method of repairing dental caries in a tooth comprising the steps:
a) drilling the tooth;
b) acid-etching the tooth;
c) administering a dental adhesive wherein the dental adhesive comprises a compound of Formula I 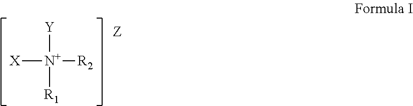
wherein X is selected from the group consisting of —R₄—O—C(O)C(R₅)═CH₂, —CH₂—CH═CH₂, and 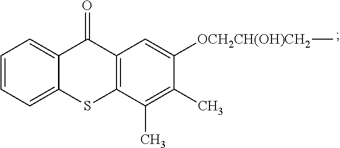
wherein Y is selected from the group consisting of hydrogen, methyl, and —CH₂—CH═CH₂;
or X and Y together with the nitrogen atom to which they are attached form a five membered ring with X and Y having the structure —CH₂—C(R₆)H—C(R₇)H—CH₂—;
wherein Z is a counterion sufficient to balance the charge of the monomer;
wherein R₁ is selected from the group consisting of hydrogen and C₁₋₂alkyl;
wherein R₂ is selected from the group consisting of hydrogen and C₁₋₂alkyl;
wherein R₄ is C₂₋₃alkylene;
wherein R₅ is selected from the group consisting of hydrogen and methyl;
wherein R₆ is an ethylene, acrylate or methacrylate group; and
wherein R₇ is an ethylene, acrylate or methacrylate group.

20. A use of a compound of Formula I for preparation of a medicament for treatment of tooth decay 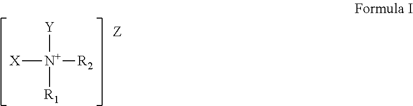
wherein X is selected from the group consisting of —R₄—O—C(O)C(R₅)═CH₂, —CH₂—CH═CH₂, and 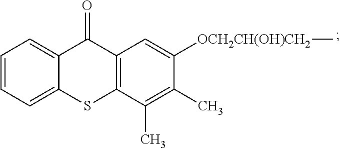
wherein Y is selected from the group consisting of hydrogen, methyl, and —CH₂—CH═CH₂;
or X and Y together with the nitrogen atom to which they are attached form a five membered ring with X and Y having the structure —CH₂—C(R₆)H—C(R₇)H—CH₂—;
wherein Z is a counterion sufficient to balance the charge of the monomer;
wherein R₁ is selected from the group consisting of hydrogen and C₁₋₂alkyl;
wherein R₂ is selected from the group consisting of hydrogen and C₁₋₂alkyl;
wherein R₄ is C₂₋₃alkylene;
wherein R₅ is selected from the group consisting of hydrogen and methyl;
wherein R₆ is an ethylene, acrylate or methacrylate group; and
wherein R₇ is an ethylene, acrylate or methacrylate group.

21. The use of claim 20, wherein the medicament comprises an acid-etching solution, a primer, an adhesive or a cement mixture, or mixtures thereof.

22. A use of a compound of Formula I for treatment of tooth decay 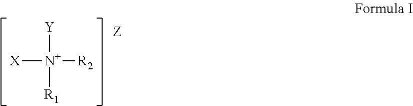
wherein X is selected from the group consisting of —R₄—O—C(O)C(R₅)═CH₂, —CH₂—CH═CH₂, and 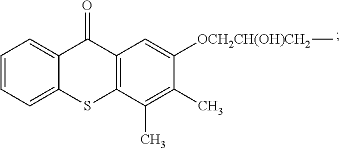
wherein Y is selected from the group consisting of hydrogen, methyl, and —CH₂—CH═CH₂;
or X and Y together with the nitrogen atom to which they are attached form a five membered ring with X and Y having the structure —CH₂—C(R₆)H—C(R₇)H—CH₂—;
wherein Z is a counterion sufficient to balance the charge of the monomer;
wherein R₁ is selected from the group consisting of hydrogen and C₁₋₂alkyl;
wherein R₂ is selected from the group consisting of hydrogen and C₁₋₂alkyl;
wherein R₄ is C₂₋₃alkylene;
wherein R₅ is selected from the group consisting of hydrogen and methyl;
wherein R₆ is an ethylene, acrylate or methacrylate group; and
wherein R₇ is an ethylene, acrylate or methacrylate group;
wherein the compound of Formula I is incorporated into a dental adhesive.
